(19) United States
(12) Patent Application Publication
(10) Pub. No.: US 2020/0038383 A1
(43) Pub. Date: Feb. 6, 2020

(54) IMMEDIATE-RELEASE ABUSE DETERRENT COMPOSITIONS OR MEDICAMENTS FOR TREATING PAIN, ADD, ADHD AND OTHER SYNDROMES OR DISORDERS

(71) Applicant: KEMPHARM, INC., Coralville, IA (US)

(72) Inventors: Travis Mickle, Celebration, FL (US); Sven M. Geunther, Coralville, IA (US); Bindu Bera, Blacksburg, VA (US); Christopher Lauderback, Lovettsville, VA (US); Jessica Sims, Orlando, FL (US); Corinna Wetzel, Orlando, FL (US)

(21) Appl. No.: 16/341,636

(22) PCT Filed: Oct. 14, 2017
(86) PCT No.: PCT/US2017/056691

§ 371 Date: Apr. 12, 2019

Related U.S. Application Data
(60) Provisional application No. 62/408,698, filed on Oct. 14, 2016.

(51) Int. Cl. A61K 31/439; A61K 47/34

(52) U.S. Cl. CPC A61K 31/439; A61K 47/34

(57) ABSTRACT

The presently described technology provides one or more compositions, preferably one or more immediate-release profile compositions, comprising aryl carboxylic acids chemically conjugated to hydrocodone (morphinan-6-one, 4,5-alpha-epoxy-3-methoxy-17-methyl), or chemically conjugated to hydromorphone (4,5,α-epoxy-3-hydroxy-17-methyl morphinan-6-one), in combination with at least one gel forming polymer; at least one disintegrant; and at least one surfactant to form novel compositions which have a decreased potential for abuse. The hydrocodone conjugate can also be combined with an analgesic, such as acetaminophen, to form a combinatorial composition that includes at least one gel forming polymer; at least one disintegrant; and at least one surfactant. The present technology also provides pharmaceutical kits and methods of synthesizing conjugates of the present technology.

### RELATED APPLICATIONS

This application claims priority to and benefit of U.S. provisional patent application No. 62/408,698, filed Oct. 14, 2016, which is herein incorporated by reference in its entirety.

### FEDERALLY SPONSORED RESEARCH OR DEVELOPMENT

[Not Applicable]

### BACKGROUND OF THE INVENTION

Opioids are highly effective as analgesics and are commonly prescribed for the treatment of acute and chronic pain. They are also commonly used as antitussives. The opioids, however, also produce euphoria and are highly addictive. As a result they are often abused with far reaching social and health related consequences. Examples of such opioids include, but are not limited to, hydromorphone, hydrocodone, among others.

Because of the inherent potential for abuse, it is desirable that any pharmaceutical composition containing an opioid agonist be made as abuse-resistant or abuse-deterrent as practical. Illicit users often will attempt to circumvent the immediate or extended release properties of these dosage forms by injecting or otherwise misusing the product in order to achieve an immediate availability or bioavailability of the opioid agonist.

Despite their addictive properties and the potential for abuse, morphine-like drugs, particularly, codeine and hydrocodone, have been routinely prescribed as treatment for severe acute and chronic pain in recent decades. This is, in part, because there are no alternatives to relieve severe pain, that is resistant to other less potent analgesics, such as non-steroidal anti-inflammatory drugs (NSAIDS) or centrally acting analgesics, such as acetaminophen or tramadol. In this regard, there is still a need to decrease the abuse potential of current opioid compositions or medicaments. Thus far, approaches taken, unfortunately, have not solved the problem.

Hydrocodone is an opioid analgesic and antitussive and occurs as fine, white crystals or as crystalline powder. Hydrocodone is a semisynthetic narcotic analgesic prepared from codeine with multiple actions qualitatively similar to those of codeine. It is mainly used for relief of moderate to moderately severe pain. Additionally, it is used as an antitussive in cough syrups and tablets.

Hydromorphone (4,5-α-epoxy-3-hydroxy-17-methyl morphinan-6-one) is a hydrogenated ketone of morphine that is used as a centrally acting opioid analgesic and antitussive. Hydromorphone is a semisynthetic narcotic analgesic prepared from morphine that possesses multiple actions qualitatively similar to those of morphine and is used in medicine as an alternative to morphine. It is mainly used for relief of pain and as a narcotic antitussive for cases of dry, painful coughing. Hydromorphone interacts predominantly with the opioid receptors in the central nervous system (CNS). Its analgesic properties are primarily due to agonist activity at the μ-opioid receptor. Hydromorphone is also a partial agonist of the δ-opioid receptor and an agonist of the κ-opioid receptor. Additionally, hydromorphone exhibits antitussive properties by suppressing the cough reflex in the medullary cough center of the brain.

Patients taking opioid analgesics such as hydrocodone and hydromorphone for pain and/or cough relief can become unintentionally addicted. As tolerance to the opioids develops, higher amounts of the drug are needed to alleviate the symptoms and generate the sense of wellbeing initially achieved with the prescribed dose. This leads to dose escalation, which, if left unchecked, can lead rapidly to addiction. In some cases patients have become addicted in as little as thirty days.

### BRIEF SUMMARY OF THE INVENTION

The present technology provides one or more compositions, comprising, for example, at least one conjugate of hydrocodone or hydromorphone, wherein the term conjugate comprises aryl carboxylic acids or derivatives thereof chemically conjugated to hydrocodone (morphinan-6-one, 4,5-alpha-epoxy-3-methoxy-17-methyl), or chemically conjugated to hydromorphone (4,5,α-epoxy-3-hydroxy-17-methyl morphinan-6-one); at least one gel forming polymer; at least one disintegrant; and at least one surfactant to form novel abuse deterrent compositions which have a decreased potential for abuse or misuse. In at least one aspect of the present technology, the compositions have an immediate release profile while still maintaining abuse-deterrent properties.

In a further aspect of the present technology, there is provided at least one composition comprising at least one conjugate and at least one gel forming polymer, at least one surfactant, and at least one disintegrant. The conjugate may be benzhydrocodone (benzoate-hydrocodone), or a pharmaceutical salt thereof, wherein benzhydrocodone has the following structure:

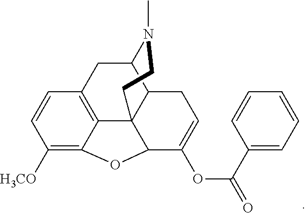

In at least one aspect of the present technology, the pharmaceutical salt of the benzhydrocodone is benzoate-hydrocodone hydrochloride (Bz-HC HCl) and has the following structure:

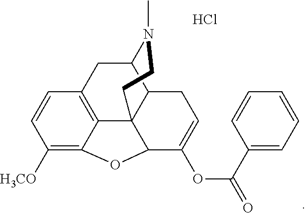

In another aspect of the practice of the present technology, there is provided at least one conjugate, at least one gel forming polymer; at least one disintegrant; and at least one surfactant, wherein the conjugate may be asalhydromorphone (3,6,-di-aspirin-hydromorphone) or a pharmaceutical salt thereof, having the following structure:

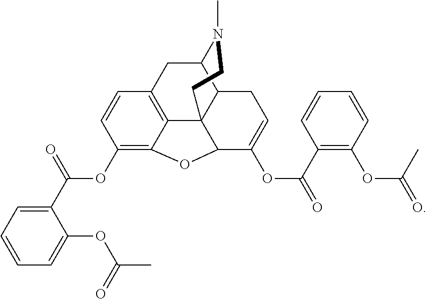

In at least one aspect of the present technology, the pharmaceutical salt of the asalhydromorphone conjugate is asalhydromorphone hydrochloride having the following structure:

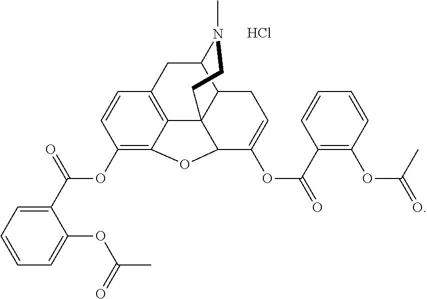

In a further embodiment, the present technology provides a composition comprising at least one conjugate, wherein the conjugate is benzhydrocodone, derivatives thereof, or a combination thereof, at least one gel forming polymer, wherein the gel forming polymer comprises one or more of polyethylene oxide, hydroxypropyl methyl cellulose, or carbomers; at least one disintegrant, wherein the disintegrant comprises one or more of sodium starch glycolate, starch, crospovidone, croscarmelose sodium, derivatives thereof, or combinations thereof; and at least one surfactant, wherein the surfactant comprises one or more of sodium lauryl sulfate, poloxamer, sorbitan monoesters, glyceryl monooleates, derivatives thereof, or combinations thereof.

In a further embodiment, the present technology provides a composition comprising at least one conjugate, wherein the conjugate is benzhydrocodone, derivatives thereof, or a combination thereof, at least one gel forming polymer, wherein the gel forming polymer is selected from the group consisting of polyethylene oxide, hydroxypropyl methyl cellulose, and carbomers; at least one disintegrant, wherein the disintegrant is selected from the group consisting of sodium starch glycolate, starch, crospovidone, croscarmelose sodium, derivatives thereof, and combinations thereof; and at least one surfactant, wherein the surfactant is selected from the group consisting of sodium lauryl sulfate, poloxamer, sorbitan monoesters, glyceryl monooleates, derivatives thereof, such as alternative salt forms, and combinations thereof.

In a further embodiment, the present technology provides a composition comprising at least one conjugate, wherein the conjugate is asalhydromorphone, derivatives thereof, or a combination thereof, at least one gel forming polymer, wherein the gel forming polymer comprises one or more of polyethylene oxide, hydroxypropyl methyl cellulose, or carbomers; at least one disintegrant, wherein the disintegrant comprises one or more of sodium starch glycolate, starch, crospovidone, croscarmelose sodium, derivatives thereof, or combinations thereof; and at least one surfactant, wherein the surfactant comprises one or more of sodium lauryl sulfate, poloxamer, sorbitan monoesters, glyceryl monooleates, derivatives thereof, or combinations thereof.

In a further embodiment, the present technology provides a composition comprising at least one conjugate, wherein the conjugate is asalhydromorphone, derivatives thereof, or a combination thereof, at least one gel forming polymer, wherein the gel forming polymer is selected from the group consisting of polyethylene oxide, hydroxypropyl methyl cellulose, and carbomers; at least one disintegrant, wherein the disintegrant is selected from the group consisting of sodium starch glycolate, starch, crospovidone, croscarmelose sodium, derivatives thereof, and combinations thereof; and at least one surfactant, wherein the surfactant is selected from the group consisting of sodium lauryl sulfate, poloxamer, sorbitan monoesters, glyceryl monooleates, derivatives thereof, and combinations thereof.

In an additional aspect, the present technology provides for a composition comprising: at least one conjugate, the conjugate comprising hydrocodone and at least one benzoic acid, a derivative thereof, a salt thereof, or a combination thereof; at least one gel forming polyethylene oxide; at least one disintegrant, wherein the disintegrant consists essentially of crospovidone, derivatives thereof, or combinations thereof; and at least one surfactant, wherein the surfactant consists essentially of sodium lauryl sulfate, derivatives thereof, or combinations thereof.

In an additional aspect, the present technology provides for a composition comprising: at least one conjugate, the conjugate comprising hydrocodone and at least one benzoic acid, a derivative thereof, a salt thereof, or a combination thereof; at least one gel forming polyethylene oxide; at least one disintegrant, wherein the disintegrant consists essentially of crospovidone, derivatives thereof, or combinations thereof; and at least one surfactant, wherein the surfactant is sodium lauryl sulfate, derivatives thereof, or combinations thereof.

In an additional aspect, the present technology provides for a composition comprising: at least one conjugate, the conjugate comprising hydrocodone and at least one benzoic acid, a derivative thereof, a salt thereof, or a combination thereof; at least one gel forming polyethylene oxide; at least one disintegrant, wherein the disintegrant is crospovidone, derivatives thereof, or combinations thereof; and at least one surfactant, wherein the surfactant consists essentially of sodium lauryl sulfate, derivatives thereof, or combinations thereof.

In an additional aspect, the present technology provides for a composition comprising: at least one conjugate, the conjugate comprising hydromorphone and at least one benzoic acid, a derivative thereof, a salt thereof, or a combination thereof; at least one gel forming polyethylene oxide; at least one disintegrant, wherein the disintegrant consists essentially of crospovidone, derivatives thereof, or combinations thereof; and at least one surfactant, wherein the surfactant consists essentially of sodium lauryl sulfate, derivatives thereof, or combinations thereof.

In an additional aspect, the present technology provides for a composition comprising: at least one conjugate, the conjugate comprising hydromorphone and at least one benzoic acid, a derivative thereof, a salt thereof, or a combination thereof; at least one gel forming polyethylene oxide; at least one disintegrant, wherein the disintegrant consists essentially of crospovidone, derivatives thereof, or combinations thereof; and at least one surfactant, wherein the surfactant is sodium lauryl sulfate, derivatives thereof, or combinations thereof.

In an additional aspect, the present technology provides for a composition comprising: at least one conjugate, the conjugate comprising hydromorphone and at least one benzoic acid, a derivative thereof, a salt thereof, or a combination thereof; at least one gel forming polyethylene oxide; at least one disintegrant, wherein the disintegrant is crospovidone, derivatives thereof, or combinations thereof; and at least one surfactant, wherein the surfactant consists essentially of sodium lauryl sulfate, derivatives thereof, or combinations thereof.

In a further aspect, the present technology provides a composition comprising a combination of at least one conjugate, the conjugate comprising benzhydrocodone, a salt thereof, or a combination thereof, and acetaminophen; at least one gel forming polymer, at least one surfactant, and at least one disintegrant.

In a still further aspect, the present technology provides a composition comprising a combination of at least one conjugate, the conjugate comprising benzhydrocodone, a salt thereof, or a combination thereof, and acetaminophen; at least one gel forming polymer, wherein the gel forming polymer comprises one or more of polyethylene oxide, hydroxypropyl methyl cellulose, or carbomers; at least one disintegrant, wherein the disintegrant comprises one or more of sodium starch glycolate, starch, crospovidone, croscarmelose sodium, derivatives thereof, or combinations thereof; and at least one surfactant, wherein the surfactant comprises one or more of sodium lauryl sulfate, poloxamer, sorbitan monoesters, glyceryl monooleates, derivatives thereof, or combinations thereof.

In a still further aspect, the present technology provides a composition comprising a combination of at least one conjugate, the conjugate comprising benzhydrocodone, a salt thereof, or a combination thereof, and acetaminophen; at least one gel forming polyethylene oxide; at least one disintegrant, wherein the disintegrant consists essentially of crospovidone, derivatives thereof, or combinations thereof; and at least one surfactant, wherein the surfactant consists essentially of sodium lauryl sulfate, derivatives thereof, or combinations thereof. In some embodiments, the composition further comprises at least one binder. In some embodiments, the binder is povidone.

In a still further aspect, the present technology provides a composition comprising a combination of at least one conjugate, the conjugate comprising benzhydrocodone, a salt thereof, or a combination thereof, and acetaminophen; at least one gel forming polyethylene oxide; at least one disintegrant, wherein the disintegrant consists essentially of crospovidone, derivatives thereof, or combinations thereof; and at least one surfactant, wherein the surfactant is sodium lauryl sulfate, derivatives thereof, or combinations thereof. In some embodiments, the composition further comprises a binder, such as povidone.

In a still further aspect, the present technology provides a composition comprising a combination of at least one conjugate, the conjugate comprising benzhydrocodone, a salt thereof, or a combination thereof, and acetaminophen; at least one gel forming polyethylene oxide; at least one disintegrant, wherein the disintegrant is crospovidone, derivatives thereof, or combinations thereof; and at least one surfactant, wherein the surfactant consists essentially of sodium lauryl sulfate, derivatives thereof, or combinations thereof. In some embodiments, the composition further comprises a binder, such as povidone.

In another embodiment, the present technology provides a composition comprising: at least one conjugate, wherein the conjugate is benzhydrocodone, derivatives thereof, or a combination of benzhydrocodone, derivatives thereof and acetaminophen, wherein benzhydrocodone has the following structure, or a pharmaceutical salt thereof:

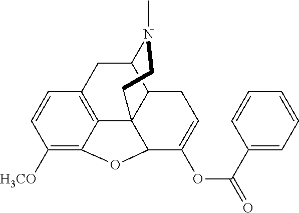

at least one gel forming polymer, wherein the gel forming polymer is selected from the group consisting of polyethylene oxide, hydroxypropyl methyl cellulose, and carbomers; at least one disintegrant, wherein the disintegrant is selected from the group consisting of sodium starch glycolate, starch, crospovidone, croscarmelose sodium, derivatives thereof, and combinations thereof; and at least one surfactant, wherein the surfactant is selected from the group consisting of sodium lauryl sulfate, poloxamer, sorbitan monoesters, glyceryl monooleates, derivatives thereof, and combinations thereof.

In an additional aspect, the present technology provides for a composition comprising: at least one conjugate, wherein the conjugate is asalhydromorphone, derivatives thereof, or a combination thereof; at least one gel forming polyethylene oxide; at least one disintegrant, wherein the disintegrant consists essentially of crospovidone, derivatives thereof, or combinations thereof; and at least one surfactant, wherein the surfactant consists essentially of sodium lauryl sulfate, derivatives thereof, or combinations thereof, wherein the composition has a ratio of conjugate to polyethylene oxide of from about 1:10 to about 3:2 w/w %.

In still another embodiment, the present technology provides a composition comprising at least one conjugate, wherein the conjugate is benzhydrocodone, derivatives thereof, or a combination thereof; at least one gel forming polyethylene oxide; at least one disintegrant, wherein the disintegrant consists essentially of crospovidone, derivatives thereof, or combinations thereof; and at least one surfactant, wherein the surfactant consists essentially of sodium lauryl sulfate, derivatives thereof, equivalents thereof, or combinations thereof, wherein the composition has a ratio of conjugate to polyethylene oxide of from about 1:5 to about 5:2 w/w %.

In another aspect, the present technology comprises a composition comprising at least one conjugate, wherein the conjugate is benzhydrocodone, derivatives thereof, or a combination thereof; acetaminophen; at least one gel forming polymer of polyethylene oxide; at least one disintegrant, wherein the disintegrant consists essentially of crospovidone, derivatives thereof, or a combination thereof, and at least one surfactant, wherein the at least one surfactant consists essentially of sodium lauryl sulfate, derivatives thereof, or combinations thereof, wherein the composition has a ratio of the combined acetaminophen and conjugate to polyethylene oxide of from about 5:1 to about 25:1 w/w %.

In additional aspects, the present technology provides a pharmaceutical kit containing a specified amount of individual doses containing an amount of a composition comprising at least one conjugate selected from the group consisting of benzhydrocodone and asalhydromorphone, or a combination of acetaminophen and a conjugate of benzhydrocodone, at least one gel forming polymer; at least one disintegrant; and at least one surfactant.

The following aspects of the presently claimed and described technology will be further understood and appreciated by those skilled in the art based upon the attendant drawings and detailed description below, which is provided herein in a non-limiting manner.

### BRIEF DESCRIPTION OF SEVERAL VIEWS OF THE DRAWINGS

FIG. 1. PK profile graph of plasma concentrations of hydrocodone released from Bz-HC (benzhydrocodone), YYFFI-HC (Tyr-Tyr-Phe-Phe-Ile-Hydrocodone) and Diglycolate-HC over time upon oral administration in rats.

FIG. 2. PK profile graph of plasma concentrations of active metabolite hydromorphone over time upon oral administration of Bz-HC, YYFFI-HC, and Diglycolate-HC in rats.

FIG. 3. PK profile graph of plasma concentrations of hydrocodone released from Bz-HC and Adipate-HC over time upon intranasal administration in rats.

FIG. 4. PK profile graph of plasma concentrations of active metabolite hydromorphone over time upon intranasal administration of Bz-HC and Adipate-HC in rats.

FIG. 5. PK profile graph of plasma concentrations of hydrocodone released from Bz-HC, Nicotinate-HC and Hydrocodone.BT over time upon oral administration in rats.

FIG. 6. PK profile graph of plasma concentrations of active metabolite hydromorphone over time upon oral administration of Bz-HC, Nicotinate-HC and Hydrocodone BT in rats.

FIG. 7. PK profile graph of plasma concentrations of hydrocodone released from Bz-HC, 2-ABz-HC and Hydrocodone BT over time upon oral administration in rats.

FIG. 8. PK profile graph of plasma concentrations of active metabolite hydromorphone over time upon oral administration of Bz-HC, 2-ABz-HC and Hydrocodone BT in rats.

FIG. 9. Synthesis diagrams of conjugates of hydrocodone.

FIG. 9A depicts the synthesis of benzoate-hydrocodone (Bz-HC).

FIG. 9B depicts the synthesis of nicotinate-hydrocodone (Nicotinate-HC).

FIG. 9C depicts the synthesis of 2-aminobenzoate-hydrocodone (2-ABz-HC).

FIG. 9D depicts the synthesis of salicylate-hydrocodone.

FIG. 10. PK profile graph of plasma concentrations of intact Bz-HC, active metabolite hydromorphone and of hydrocodone released from Bz-HC over time upon oral administration in rats.

FIG. 11. PK profile graph of plasma concentrations of hydrocodone released from Bz-HC and hydrocodone BT over time upon oral administration in dogs.

FIG. 12. PK profile graph of plasma concentrations of active metabolite hydromorphone over time upon oral administration of Bz-HC and hydrocodone.BT in dogs.

FIG. 13. PK profile graph of plasma concentrations of intact Bz-HC and of hydrocodone released from Bz-HC over time upon oral administration in dogs.

FIG. 14. PK profile graph of plasma concentrations of intact Bz-HC, active metabolite hydromorphone and of hydrocodone released from Bz-HC over time upon intravenous administration in rats at 0.30 mg/kg.

FIG. 15. PK profile graph of plasma concentrations of hydrocodone released from Bz-HC over time upon oral administration in rats at six different dosages.

FIG. 16. PK profile graph of plasma concentrations of active metabolite hydromorphone over time upon oral administration of Bz-HC in rats at six different dosages.

FIG. 17. Pharmacokinetic profile of released hydromorphone (HM) in the plasma of rats that were dosed intranasally with doses of 3,6-di-aspirin-HM and HM equimolar to 2.0 mg/kg of hydromorphone.

FIG. 18. Pharmacokinetic profile of released hydromorphone (HM) in the plasma of rats that were dosed intravenously with doses of 3,6-di-aspirin-HM and HM equimolar to 0.2 mg/kg of hydromorphone.

FIG. 19. Area under the curve (AUC) of released hydromorphone (HM) in the plasma of rats that were dosed orally with escalating equimolar doses of HM and 3,6-di-aspirin-HM.

FIG. 20. Area under the curve (AUC) and peak plasma concentrations (Cₘₐₓ) in the plasma of rats that were dosed orally with equimolar doses of HM, untampered 3,6-di-aspirin-HM, and hydrolytic breakdown products of 3,6-di-aspirin-HM.

FIG. 21. Example synthetic schemes for the synthesis of some of the hydromorphone prodrugs of the present technology.

FIG. 22a. PK profile graph of plasma concentrations of hydrocodone released from Bz-HC.HCl/APAP (6.67 mg/325 mg) and HB/APAP over a complete time course upon oral administration of three single doses in recreational drug users.

FIG. 22b. PK profile graph of plasma concentrations of hydrocodone released from Bz-HC.HCl/APAP (6.67 mg/325 mg) and HB/APAP over a first 3 hours of post-dose upon oral administration of three single doses in recreational drug users.

FIG. 23a. PK profile graph of plasma concentrations of hydromorphone released from Bz-HC.HCl/APAP (6.67 mg/325 mg) and HB/APAP over a complete time course upon oral administration of three single doses in recreational drug users.

FIG. 23b. PK profile graph of plasma concentrations of hydromorphone released from Bz-HC.HCl/APAP (6.67 mg/325 mg) and HB/APAP over a first 3 hours of post-dose upon oral administration of three single doses in recreational drug users.

FIG. 24 is a graph showing the dissolution rates for benzhydrocodone (30 mg) formulations using a proof of concept discriminating dissolution method.

FIG. 25 is a graph showing the dissolution rate for a 30 mg benzhydrocodone formulation using a release dissolution method.

### DETAILED DESCRIPTION OF THE INVENTION

As used herein, “APAP” refers to acetaminophen.

As used herein, “immediate release or immediate release profile” means the dissolution of about 50% of the contained active within a product within 10 minutes when using the defined discriminating conditions utilizing USP apparatus 2 at 50 rpm in a 900 mL bath of 0.1N HCl with 0.01% CTAB at 37° C. with 2S sinkers. Alternatively, as used herein “immediate release” or “immediate release profile” means the dissolution of as much as 75% of the contained active within a product in 10 minutes when using the defined non-discriminating conditions utilizing USP apparatus 2 at 50 rpm in a 900 mL bath of 0.1N HCl with 0.01% CTAB at 37° C. with 4S sinkers.

As used herein, “dissolution discriminating method” refers to a method that uses the defined discriminating conditions utilizing USP apparatus 2 at 50 rpm in a 900 mL bath of 0.1N HCl with 0.01% CTAB at 37° C. with 2S sinkers.

As used herein “non-discriminating method” refers to a method that uses the defined non-discriminating conditions utilizing USP apparatus 2 at 50 rpm in a 900 mL bath of 0.1N HCl with 0.01% CTAB at 37° C. with 4S sinkers.

The use of the term “dose” means the total amount of a drug or active component taken each time by an individual.

The term “unit dose form” here means a single entity of a solid therapeutic dosage form (e.g., 1 capsule, 1 tablet) or a single volume dispensed from a non-solid dosage form (e.g., 5 mL of a liquid or syrup).

As used herein, “consisting essentially of” means only those specified materials or steps that follow it that make up the composition/formulation, as well as other materials that do not materially affect the basic and novel characteristic(s).

As used herein, “abuse deterrent properties” means properties imparted to the conjugate formulation by PEO that result in the formation of a gel when put into contact with water or other solvents. When the formulation is crushed or pulverized, and administered intranasally, this gel forms inside the nose making it more difficult to snort the full dose or slowing drug absorption in the nasal cavity when compared to formulations that do not form a gel. The gel also deters intravenous abuse by making it difficult to put into a syringe for injection. Abuse deterrent properties may also mean properties imparted to the conjugate formulation by the surfactant that may irritate the nasal mucous membranes or cause a burning sensation in the nose or face when administered intranasally.

“Cₘₐₓ”, used hereinafter, is a term used in pharmacokinetics that refers to the maximum (or peak) blood plasma concentration.

“Tₘₐₓ”, used hereinafter, is the term used in pharmacokinetics to describe the time at which the Cₘₐₓ is observed.

“AUC_{0-inf}”, used hereinafter, is the term to describe area under the plasma concentration versus time curve from time zero to infinity.

The presently claimed invention(s) and presently described technology include one or more abuse deterrent formulations for reducing the potential for one or more of a) parenteral abuse, b) inhalation (e.g., intranasal abuse), and/or c) oral abuse of an opioid analgesic type drug, such as hydrocodone or hydromorphone, for satisfaction of a physical or psychological dependence. In at least one embodiment, the presently described and claimed technology deters parenteral abuse by providing a pharmaceutical composition which includes at least one benzhydrocodone conjugate or at least one asalhydromorphone conjugate with one or more gel forming agents such that upon contact with a small amount (a tablespoon or less) of solvent (e.g., water), the agents swell by absorbing the solvent thereby 1) entrapping the conjugate in a gel matrix and/or 2) reducing or preventing a significant amount of the conjugate from being drawn into a syringe. It should be appreciated by those skilled in the art that this particular embodiment can be modified to provide abuse deterrence with an immediate release profile by incorporating an effective amount of a suitable disintegrant. It should also be appreciated that this particular embodiment also envisages the use of formulation technology such as the combinatorial pharmaceutical product of benzhydrocodone conjugate combined with acetaminophen, among others, while still achieving abuse deterrence in an immediate release profile manner.

In a further embodiment, the presently claimed and described technology deters nasal insufflation abuse by providing a pharmaceutical composition which includes benzhydrocodone conjugate or asalhydromorphone conjugate, with one or more mucous membrane, mucosa or mucosal tissue irritants (collectively referred to as mucous membrane irritants). In one embodiment, the mucosal tissue is nasal passageway tissue. Upon contact with a mucous membrane, the irritants induce temporary pain and/or irritation of the membranes and/or tissues to thereby deter abuse. For example, if inhaled by snorting, the mucous membrane in the nasal passageway will be irritated and result in pain to the individual.

In at least one embodiment, the presently described and claimed technology provides at least one pharmaceutical composition which includes benzhydrocodone conjugate or asalhydromorphone conjugate, such that after oral consumption of more than a typically prescribed amount of the dosage form, emesis is induced. It should be appreciated that combinatorial products with an emetic composition or medicament are also envisaged. It should also be appreciated by those skilled in the art, that in a further embodiment(s), two or more of the abuse deterrents can be combined into one composition according to the technology and practice of the presently claimed invention(s).

In another embodiment, the presently described and claimed technology involves at least one pharmaceutical composition that includes benzhydrocodone conjugate or asalhydromorphone conjugate or pharmaceutically acceptable salts thereof, with one or more gel forming agents, and one or more mucous membrane irritants or nasal passageway tissue irritants.

In a still further embodiment, the presently claimed and described technology includes, for example, at least one pharmaceutical composition, which includes at least one benzhydrocodone conjugate, or pharmaceutically acceptable salts thereof, with one or more gel forming agents as described herein. The pharmaceutical composition can further comprise one or more analgesics, such as acetaminophen, in combination with the benzhydrocodone conjugate to form a combinatorial pharmaceutical product. In one particular embodiment, the present technology can include at least one pharmaceutical composition which includes, for example, benzhydrocodone conjugate, an acetaminophen analgesic, one or more gel forming agents, one or more mucous membrane irritants and/or nasal passageway tissue irritants, and one or more emetics.

In still another aspect, the present technology includes a composition comprising at least one conjugate, the conjugate comprising benzhydrocodone, acetaminophen, optionally at least one additive or at least one antidispersive; at least one gel forming polyethylene oxide; at least one disintegrant, wherein the disintegrant consists essentially of crospovidone, derivatives thereof, or combinations thereof; and at least one surfactant, wherein the surfactant consists essentially of sodium lauryl sulfate, derivatives thereof, or combinations thereof.

In a further embodiment, the present technology comprises at least one conjugate, the conjugate comprising benzhydrocodone and at least one additive or at least one antidispersive; at least one gel forming polyethylene oxide; at least one disintegrant, wherein the disintegrant consists essentially of crospovidone, derivatives thereof, or combinations thereof; and at least one surfactant, wherein the surfactant consists essentially of sodium lauryl sulfate, derivatives thereof, or combinations thereof. In some additional embodiments, the composition further comprises at least one binder. In some embodiments the binder is povidone.

Each of the components of the pharmaceutical compositions of the present technology are described in more detail below.

A. Drugs, Compositions, Medicaments, and/or Combinatorial Compositions or Medicaments Suitable for Use with the Present Technology

In some embodiments, pharmaceutical compositions of the present technology include at least one of asalhydromorphone conjugate or benzhydrocodone conjugate, or salts thereof, as the therapeutically active ingredient. In some embodiments, the benzhydrocodone conjugate has the following structure:

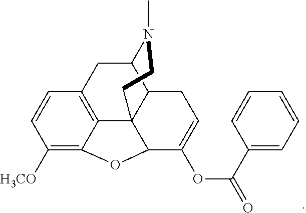

In some embodiments, the benzhydrocodone conjugate is a hydrochloride salt of benzhydrocodone having the following structure:

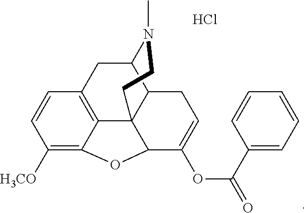

In some embodiments, the asalhydromorphone conjugate is 3,6-di-aspirin-hydromorphone having the following structure:

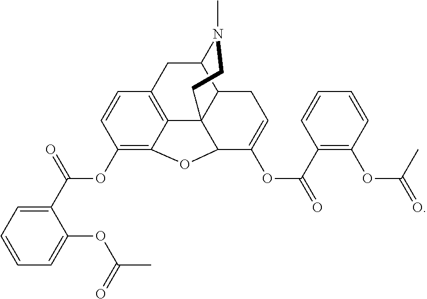

In some embodiments, the asalhydromorphone conjugate is a hydrochloride salt:

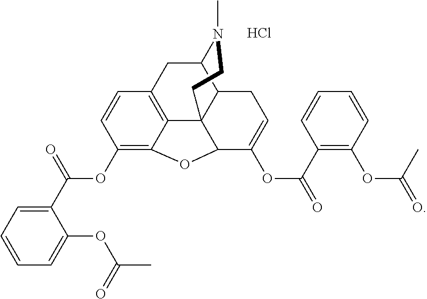

Pharmaceutical salts are known to those of skill in the art and include acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, l-lactate, d,l-lactate, d,l-malate, l-malate, d-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsufate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate (mucate), galacturonate, gallate, gentisate, glutamate, glutarate, glycerophosphate, heptanoate (enanthate), hydroxybenzoate, hippurate, phenylpropionate, iodide, xinafoate, lactobionate, laurate, maleate, mandelate, methanesufonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, undecylenate, sodium, potassium, calcium, magnesium, zinc, aluminum, lithium, cholinate, lysinium, ammonium, and tromethamine.

Typically when processed into a suitable dosage form, as described in more detail below, the active can be present in such dosage forms in an amount normally prescribed, typically about 0.1 to about 50 percent on a dry weight basis, based on the total weight of the formulation. It should also be appreciated and as described and claimed herein that conjugation of the therapeutically active ingredient to a benzoate ligand provides abuse deterrence along with the immediate release profile and abuse deterrence that is conferred by the gel-forming polymer as described herein.

Compositions of the present technology can be provided in unit dose form, with the amount of active typically being from about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.1 mg, about 1.125 mg, about 1.3 mg, about 1.4 mg, about 1.5 mg, about 1.6, about 1.7 mg, about 1.8 mg, about 1.9 mg, about 2.0 mg, about 2.1 mg, about 2.2 mg, about 2.3 mg, about 2.4 mg, about 2.5 mg, about 2.6 mg, about 2.7 mg, about 2.8 mg, about 2.9 mg, about 3.0 mg, about 3.1 mg, about 3.2 mg, about 3.3 mg, about 3.4 mg, about 3.5 mg, about 3.6 mg, about 3.7 mg, about 3.8 mg, about 3.9 mg, about 4.0 mg, about 4.1 mg, about 4.2 mg, about 4.3 mg, about 4.4 mg, about 4.5 mg, about 4.6 mg, about 4.7 mg, about 4.8 mg, about 4.9 mg, about 5 mg, about 5.1 mg to about 5.9 mg, about 6.0 mg to about 6.9 mg, about 7.0 mg to about 7.9 mg, about 8.0 mg to about 8.9 mg, about 9.0 mg to about 9.9 mg, about 10.0 mg to about 10.9 mg, about 11.0 mg to about 11.9 mg, about 12.0 mg to about 12.9 mg, about 13.0 mg to about 13.9 mg, about 14.0 mg to about 14.9 mg, about 15.0 mg to about 15.9 mg, about 16.0 mg to about 16.9 mg, about 17.0 mg to about 17.9 mg, about 18.0 mg to about 18.9 mg, about 19.0 mg to about 19.9 mg, about 20 mg and all sub-ranges in between to about 25 mg, about 25.1 mg and all subranges to about 50 mg, 51.1 mg and all subranges to about 75 mg, 75.1 mg and all subranges to about 100 mg, 100.1 mg and all subranges to about 125 mg, 125.1 mg and all subranges to about 150 mg, 150.1 mg and all subranges to about 175 mg, 175.1 mg and all subranges to about 200 mg, or potentially higher depending upon the desired pain relief and analgesic chosen. Thus, it should be appreciated by those skilled in the art that the active ingredient desired for use and practice of the present technology and the attendant claims include, but are not limited to all variations from 0.1 and up as well as all multiples thereof. In some embodiments, the active can be present in an amount from about 0.5 mg to about 25 mg. For example, in some embodiments, the active can be present in an amount from about 1 mg to about 10 mg, alternatively about 1.5 mg to about 8 mg, alternatively about 1.8 mg to about 7.5 mg, alternatively about 1.8 mg to about 7.1 mg. In some embodiments, the hydromorphone active can be present in an amount of about 1 mg to about 60 mg, alternatively about 1 mg to about 56.7 mg, alternatively about 1.8 mg to about 30 mg, alternatively about 2 mg to about 28.4, about 3.5 mg to about 15 mg, alternatively about 5 mg to about 14.2 mg, alternatively about 7.1 mg to about 12 mg, alternatively about 8 mg to about 10.6 mg. In some embodiments, the hydrocodone active can be present in an amount from about 5 mg to about 25 mg, alternatively about 5 mg to about 22.3 mg, alternatively about 7.4 mg to about 20 mg, alternatively about 6.5 mg to about 20 mg, alternatively about 10 mg to about 14.8 mg. In some combinatorial embodiments, the amount of hydrocodone active can be present in an amount from about 1.5 mg to about 8 mg, alternatively about 3 mg to about 7 mg, alternatively about 3 mg to about 6.5 mg, alternatively about 3 mg to about 6.1 mg, alternatively about 4.5 mg to about 6.1 mg, and the amount of acetaminophen active can be present in an amount from about 100 mg to about 350 mg, alternatively about 150 mg to about 325 mg, alternatively about 162.5 mg to about 325 mg, alternatively about 216.7 to about 325, alternatively about 300 to about 325 mg.

In other embodiments, a dosage form contains an appropriate amount of the conjugate to provide a therapeutic effect. In some embodiments, the conjugate can be present in the dosage in an amount of about 1 mg or higher, such as about 1 mg to about 30 mg In some embodiments, the benzhydrocodone conjugate can be present in the dosage in an amount of about 9 mg or higher, such as about 9 mg to about 30 mg. In some combinatorial embodiments comprising the benzhydrocodone conjugate and acetaminophen, the benzhydrocodone conjugate can be present in the dosage in an amount of about 1 mg or higher, such as about 1.4 mg to about 9 mg, alternatively about 4 mg to about 9 mg. In some embodiments, the asalhydromorphone conjugate can be present in the dosage in an amount of about 3.5 mg to about 32 mg, alternatively about 3.5 mg to about 20 mg, alternatively about 3.5 mg to about 19 mg, alternatively about 3.5 mg to about 16 mg. Additionally, while not intending to be a limitation, it is preferable that the unit dose form be formulated in such a manner to provide a dosing regimen that enhances abuse deterrence, such as a once a day or twice a day dosing regimen, that still achieves an immediate release profile while achieving or maintaining the desired abuse deterrent outcome.

B. Gel Forming Agents

As described above, the presently described and claimed technology can include one or more gel forming agents. The total amount of gel forming agent is typically about 3% to about 40%, alternatively about 3% to about 15%, about 20%, or about 25% on a dry weight basis of the total composition.

Suitable gel forming agents include, but are not limited to compounds that, upon contact with a solvent (e.g., water), absorb the solvent and swell, thereby forming a viscous or semi-viscous substance that significantly reduces and/or minimizes the amount of free solvent which can contain an amount of conjugate and thereby reduce the amount of conjugate which can be drawn into a syringe. The gel can also reduce the overall amount of active or active combination (or separate components of the combination) extractable with the solvent by entrapping the active in a gel matrix. The present technology also makes extraction of the active more difficult, because the conjugate in the gel matrix must be further broken down or manipulated in order to obtain the active. In one exemplary embodiment, typical gel forming agents include pharmaceutically acceptable polymers, typically hydrophilic polymers, such as hydrogels.

In some additional non-limiting embodiments, the polymers suitable for the practice of the present technology exhibit a high degree of viscosity upon contact with a suitable solvent. While not being bound by any particular theory, it is believed that the high viscosity can enhance the formation of highly viscous gels when attempts are made by an abuser to crush and dissolve the contents of a dosage form in an aqueous vehicle and inject it intravenously. More specifically, in certain preferable embodiments, but in a non-limiting manner, the polymeric material in the present technology provides a viscosity to the dosage form when it is tampered. In such embodiments, when an abuser crushes and dissolves the dosage form in a solvent (e.g., water or saline), a viscous or semi-viscous gel is formed. Again, without being bound to any particular theory, it is believed that the increase in the viscosity of the solution discourages the abuser from injecting the gel intravenously or intramuscularly by preventing the abuser from transferring sufficient amounts of the solution to a syringe to cause a desired “high” once injected.

Suitable polymers include, but are not limited to, one or more pharmaceutically acceptable polymers selected from any pharmaceutical polymer that will undergo an increase in viscosity upon contact with a solvent, but not increase in viscosity so rapidly as to hinder the disintegrant from acting to achieve an immediate release profile. Preferred polymers include polyethylene oxide, hydroxypropyl methyl cellulose and carbomers. Polyvinyl alcohol is not a preferred polymer for use herein, since the amount required to achieve sufficient gelling may be too high for an acceptable unit dose. In some preferred embodiments, the polymers can include:

a) Polyethylene Oxide

For example, in some embodiments of the presently described and claimed technology, the polymer includes polyethylene oxide. The polyethylene oxide can have an average molecular weight ranging from about 900,000 to about 7,000,000, more preferably from about 2,000,000 to about 6,000,000, and most preferably at least about 5,000,000. In one particular embodiment, the polyethylene oxide includes a high molecular weight polyethylene oxide.

In a further embodiment, the average particle size of the polyethylene oxide ranges from about 840 microns to about 2,000 microns. In another embodiment, the density of the polyethylene oxide can range from about 1.15 g/mL to about 1.26 g/mL. In a still further embodiment, the viscosity can range from about 8,800 cps to about 17,600 cps.

In some additional embodiments, the polyethylene oxide used in a directly compressible formulation of the presently described and claimed technology can be preferably a homopolymer having repeating oxyethylene groups, i.e., —(—O—CH₂—CH₂—)ₙ—, where n can range from about 2,000 to about 180,000. Also preferably, the polyethylene oxide is a commercially available and pharmaceutically acceptable homopolymer having a moisture content of no greater than about 1% by weight. Non-limiting examples of suitable, commercially available polyethylene oxide polymers include Polyox®, WSRN-1105 and/or WSR-coagulant, available from Dow chemicals.

In other exemplary embodiments, powdered polyethylene oxide polymers can contribute to a consistent particle size in a directly compressible formulation and eliminate the problems of lack of content uniformity and possible segregation.

In some embodiments, the amount of polyethylene oxide polymer in the composition has an effect on the ability to obtain an immediate release formulation. In some embodiments, an immediate release composition can be formulated using from about 3% to less than about 15% by weight of a polyethylene oxide polymer having an average molecular weight of about 5,000,000 as the gel forming polymer. Higher amounts of polyethylene oxide in the composition can lead to an extended release profile. In some embodiments, the amount of polyethylene oxide can be from about 3% to about 12% by weight, alternatively about 4% to about 10% by weight. In some embodiments of the present technology, the weight ratio of the conjugate or the combined conjugate and acetaminophen to the polyethylene oxide in the composition may be important for achieving the desired immediate release profile. In some embodiments, the weight ratio of benzhydrocodone to polyethylene oxide in the composition can be 1:6 to 5:2, alternatively 1:5 to 5:2, and can be 1:4 to 2:1, alternatively 1:4 to 3:2, alternatively 1:4 to 1:1 when an immediate release profile is desired. In some embodiments, the weight ratio of asalhydromorphone to polyethylene oxide can be 1:10 to about 3:2, alternatively 1:10 to 1:1, alternatively 1:9 to 3:4, alternatively 1:8 to 3:5 when an immediate release profile is desired. In some embodiments, the weight ratio of the combined benzhydrocodone and acetaminophen to polyethylene oxide can be 5:1 to 25:1, alternatively 7.5 to 20:1, alternatively 10:1 to 15:1 when an immediate release profile is desired.

b) Hydroxypropyl Methyl Cellulose

In at least one embodiment, the gel forming agent of the presently claimed and described technology includes (in a non-limiting manner) hydroxypropyl methyl cellulose (Hypromellose). The hydroxypropyl methyl cellulose can have a molecular weight ranging from about 10,000 to about 1,500,000, and typically from about 5000 to about 10,000, i.e., a low molecular weight hydroxypropyl methyl cellulose polymer. The specific gravity of the hydroxypropyl methyl cellulose can range from about 1.19 to about 1.31, with an average specific gravity of about 1.26 and a viscosity of about 3600 to 5600 cps. The hydroxypropyl methyl cellulose used in the exemplary formulations of the present technology can be a water-soluble synthetic polymer. Examples of suitable, commercially available hydroxypropyl methylcellulose polymers include METHOCEL® K100 LV and METHOCEL® K4M, available from Dow chemicals.

c) Carbomers

In at least one embodiment, the presently described and claimed technology includes one or more carbomers. The carbomers can have a molecular weight ranging from 700,000 to about 4,000,000,000. The viscosity of the polymer can range from about 4000 cps to about 39,400 cps. A non-limiting example of a suitable, commercially available carbomer is CARBOPOL® 971P NF, available from Noveon Pharmaceuticals.

Following the teachings set forth herein, other suitable gel forming agents for use in the practice of the present technology can include one or more of the following polymers: ethyl cellulose, cellulose acetate, cellulose acetate propionate, cellulose acetate butyrate, cellulose acetate phthalate and cellulose triacetate, cellulose ether, cellulose ester, cellulose ester ether, and cellulose, acrylic resins comprising copolymers synthesized from acrylic and methacrylic acid esters, the acrylic polymer may be selected from the group consisting of acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers, among others.

It should be appreciated by those skilled in the art that any of the above described polymers can be combined together or combined with other suitable polymers, and such combinations are within the scope of the presently described and claimed invention.

C. Abuse Deterrency of the Abuse Deterrent Gel

In one or more embodiments of the formulations of the present technology, the gel forming polymer assists in imparting one or more abuse deterrent properties to the formulations. Incorporating the gel forming polymer into the formulation results in the formation of a gel when put into contact with water or other solvents. When the formulation is crushed or pulverized, and administered intranasally, this gel forms inside the nose making it more difficult to snort the full dose or slowing drug absorption in the nasal cavity when compared to formulations that do not form a gel. The gel also deters intravenous abuse by making it difficult to put into a syringe for injection. Additionally, it should also be appreciated by those skilled in the art that the above described gel forming agents can be further optimized as necessary or desired in terms of viscosity, molecular weight, etc.

D. Mucous Membrane Irritants and/or Nasal Passageway Tissue Irritants

As described above, the presently described and claimed technology can include one or more mucous membrane irritants and/or nasal passageway tissue irritants. In at least one embodiment of the present technology, suitable mucous membrane irritants and/or nasal passageway tissue irritants can include in a non-limiting manner compounds that are generally considered pharmaceutically inert, yet can induce irritation. Such compounds include, but are not limited to surfactants. In one exemplary embodiment, suitable surfactants include, but are not limited to, sodium lauryl sulfate, poloxamer, sorbitan monoesters, and glyceryl monooleates. Additional useful irritants may include sucrose laurate, dodecyl trimethyl ammonium bromide (DTAB), sodium dodecylbenzenesulphonate (DBS), sodium secondary dodecan sulfonate (SDS), sodium laurate, cocamidopropyl betaine (CAPB), malic acid, 2-hydroxybutyric acid, glycolic acid, lactic D(−)-lactic acid, L(+)-lactic acid, citric acid

Other suitable compounds are believed to be within the knowledge of a practitioner skilled in the relevant art, and can be found in the Handbook of Pharmaceutical Excipients, 7th Ed. (2012), the entire content of which is hereby incorporated by reference.

In at least one further embodiment of the presently described and claimed technology, the irritant can be present in amount of from about 1 percent to about 20 percent by weight on a solid basis, preferably about 1 percent to about 10 percent by weight on a solid basis. In other embodiments, the amount of irritant can be present in an amount of about 5 percent to about 15 percent by weight. In still other embodiments, the irritant can be present in an amount of at least about 2 percent by weight. In yet further embodiments, the irritant can be present in an amount from about 1 percent to about 5 percent by weight. In other embodiments, the amount of irritant can be present in an amount from about 2 to about 5 percent by weight. In yet other embodiments, the amount of irritant can be present in an amount from about 2 percent to about 12 percent by weight, alternatively about 3 percent to about 10 percent by weight.

In certain non-limiting embodiments, and not to be limited by any particular theory, it is believed that the irritant can deter abuse of a dosage form when a potential abuser tampers with a dosage form of the presently described and claimed technology. Specifically, in such embodiments, when an abuser crushes the dosage form, the irritant is exposed. The irritant discourages insufflation of the crushed dosage form by inducing pain and/or irritation of the abuser's mucous membrane and/or nasal passageway tissue. In an exemplary non-limiting embodiment, it is believed that the irritant discourages inhalation (e.g., via snorting through the nose) by inducing pain and/or irritation of the abuser's nasal passageway tissue. It should be appreciated by one skilled in the art that in some embodiments of the present technology, inhalation is also discouraged by utilizing lower dosage forms that minimize the ability to snort a volume large enough to make its way down the throat.

In at least one additional exemplary and non-limiting embodiment, the presently described and claimed technology includes one or more mucous membrane irritants to cause irritation of mucous membranes located anywhere on or in the body, including membranes of the mouth, eyes, and intestinal tract. It is further believed that such compositions can deter abuse via oral, intra-ocular or rectal or vaginal routes.

Additionally, it should be appreciated by those skilled in the art that the above-described irritants can be further optimized as necessary or desired in terms of concentration, irritation severity, etc.

E. Other Ingredients

The presently described and claimed technology can also optionally include other ingredients to enhance dosage form manufacture from a pharmaceutical composition of the present technology and/or alter the release profile of a dosage form including a pharmaceutical composition, medicament, drug, drug composition, or combinatorial medicament or composition of the present technology.

For example, some embodiments of the presently described and claimed technology can include one or more pharmaceutically acceptable fillers/diluents. In at least one embodiment, AVICEL® PH (Microcrystalline cellulose) is a filler used in the formulation. The AVICEL® PH can have an average particle size ranging from about 20 μm to about 200 μm, preferably about 100 μm. The density ranges from about 1.512 g/cm³ to about 1.668 g/cm³. The AVICEL® PH should have a molecular weight of about 36,000. Although not wanting to be bound by any particular theory or application of the present technology, it is believed that AVICEL® PH effectiveness is optimal when it is present in an amount of from about 10 percent to 65 percent, by weight on a solid basis, of the formulation. Typical fillers can be present in amounts from about 10 percent to 65 percent by weight, alternatively about 25 percent to about 65 percent on a dry weight basis. Other ingredients can include sugars and/or polyols.

Other ingredients for use in the practice of the present technology can also include dibasic calcium phosphate having a particle size of about 75 microns to about 425 microns and a density of about 0.5 g/mL to about 1.5 g/mL, as well as calcium sulfate having a particle size of about 1 micron to about 200 microns and a density of about 0.6 g/mL to about 1.3 g/mL, and mixtures thereof. Further, lactose having a particle size of about 20 microns to about 400 microns and a density of about 0.3 g/mL to about 0.9 g/mL can also be included.

In some embodiments, the formulations of the present technology can further include one or more binders. Binders may be selected from a wide range of materials such as hydroxypropylmethylcellulose, ethylcellulose, or other suitable cellulose derivatives, povidone, acrylic and methacrylic acid co-polymers, pharmaceutical glaze, gums, milk derivatives, such as whey, starches, and derivatives, as well as other conventional binders known to persons working in the art. In some embodiments, the binder is povidone having a molecular weight of about 2,5000 to about 50,000, a particle size distribution of about 50 microns to about 200 microns, and a bulk density of about 0.29 to about 0.39 g/mL. In some embodiments, suitable amounts of binder can be about 0.1% to about 5%, alternatively, about 0.4% to about 2% by weight.

In some non-limiting embodiments of the present technology, the fillers also function as binders in that they not only impart cohesive properties to the material within the formulation, but can also increase the bulk weight of a directly compressible formulation (as described below) to achieve an acceptable formulation weight for direct compression. In some additional non-limiting embodiments, additional fillers need not provide the same level of cohesive properties as the binders selected, but can be capable of contributing to formulation homogeneity and resist segregation from the formulation once blended. Further, preferred fillers do not have a detrimental effect on the flowability of the composition or dissolution profile of the formed tablets.

In at least one further embodiment, the presently described and claimed technology can include one or more pharmaceutically acceptable disintegrants. Such disintegrants are known to a skilled artisan. In the present technology, disintegrants can include, but are not limited to, sodium starch glycolate (EXPLOTAB®) having a particle size of about 104 microns and a density of about 0.756 g/mL, starch (e.g., Starch 21) having a particle size of about 2 microns to about 32 microns and a density of about 0.462 g/mL, crospovidone having a particle size of about 400 microns or less and a density of about 1.22 g/mL, and croscarmellose sodium (AC-DI-SOL®) having a particle size of about 37 microns to about 73.7 microns and a density of about 0.529 g/mL. The disintegrant selected should contribute to the compressibility, flowability and homogeneity of the formulation. Further, without being bound by any particular theory, it is believed that the disintegrant can minimize segregation and provide an immediate release profile to the formulation. Thus, in at least some embodiments, the disintegrant(s) of the present technology are present in an amount from about 2 percent to about 50 percent, alternatively about 2 percent to about 25 percent, alternatively about 4 percent to about 20 percent, alternatively about 7 percent to about 18 percent, alternatively about 7 percent to about 45 percent, alternatively about 10 percent to about 40 percent, by weight on a solid basis of the directly compressible formulation.

In one embodiment, the present invention can include one or more pharmaceutically acceptable glidants, including but not limited to colloidal silicon dioxide. In one embodiment, colloidal silicon dioxide (Cab-O-Sil®) having a density of about 0.029 to about 0.040 g/mL can be used to improve the flow characteristics of the formulation. Such glidants can be provided in an amount of from about 0.1 to about 1 percent by weight of the formulation on a solid basis. It will be understood, based on this invention, however, that while colloidal silicon dioxide is one particular glidant, other glidants having similar properties which are known or to be developed could be used, provided they are compatible with other excipients and the active ingredient in the formulation, and do not significantly affect the flowability, homogeneity and compressibility of the formulation.

Still further, in at least one embodiment, the presently described and claimed technology can include one or more pharmaceutically acceptable lubricants, including but not limited to magnesium stearate. In at least one exemplary embodiment, the magnesium stearate has a particle size of about 450 to microns about 550 microns and a density of about 1.00 g/mL to about 1.80 g/mL. In at least one further embodiment, magnesium stearate can contribute to reducing friction between a die wall and a pharmaceutical composition of the present invention during compression and can ease the ejection of the tablets, thereby facilitating processing. In some additional embodiments, the lubricant resists adhesion to punches and dies and/or aids in the flow of the powder in a hopper and/or into a die. In an exemplary embodiment of the present technology, magnesium stearate having a particle size of from about 5 microns to about 50 microns and a density of from about 0.1 g/mL to about 1.1 g/mL is used in a pharmaceutical composition. In certain exemplary embodiments of the present technology, a lubricant should make up from about 0.1 percent to about 2 percent by weight of the formulation on a solids basis. Suitable lubricants are stable and do not polymerize within the formulation once combined. Other lubricants known in the art or to be developed which exhibit acceptable or comparable properties include stearic acid, hydrogenated oils, sodium stearyl fumarate, polyethylene glycols, and Lubritab®.

In certain additional exemplary embodiments, the most important criteria for selection of the excipients are that the excipients should achieve good content uniformity and release the active ingredient as desired. The excipients, by having excellent binding properties, and homogeneity, as well as good compressibility, cohesiveness and flowability in blended form, minimize segregation of powders in the hopper during direct compression.

In another exemplary embodiment, the presently described and claimed technology can include at least one opioid antagonist in addition to the other ingredients, or as a substitute for one of the other abuse deterrent ingredients of a formulation of the present technology. Suitable antagonists include, but are not limited to naloxone. It is believed and understood by those skilled in the art that naloxone has no action when taken orally, and will not interfere with the pharmacologic action of an opioid agonist. However, when given by injection naloxone can have profound antagonistic action to opioid agonists. An appropriate antagonist can be used in combination with one or more of the compositions or medicaments, gel forming agents, mucous membrane irritants and/or nasal passageway tissue irritants, or emetics in the present technology. An appropriate antagonist can also be used as a substitute for one or more of gel forming agents, mucous membrane irritants and/or nasal passageway tissue irritants, or emetics in the present technology or as a component of combinatorial compositions or medicaments of the present technology. Suitable opioid receptor antagonists can include but are not limited to the antagonists described in U.S. Pat. Nos. 6,559,159 and 6,375,957, the entire content of which are hereby incorporated by reference.

F. Dosage Forms of the Present Technology

A pharmaceutical composition of the presently described and claimed technology includes at least one conjugate of hydrocodone or hydromorphone, one or more of gel forming agents, mucous membrane irritants and/or nasal passageway tissue irritants, and emetics, and optionally other ingredients, and can be suitably modified and processed to form a dosage form of the present technology.

Suitable formulations and dosage forms of the present technology include but are not limited to powders, caplets, pills, suppositories, gels, soft gelatin capsules, capsules, sachets, lozenges, troches, slurries, suspensions, solutions, oral films, and/or compressed tablets manufactured from a pharmaceutical composition or medicament of the present technology. The dosage forms can be any shape, including regular or irregular shape, depending upon the needs of the artisan.

Compressed tablets including the pharmaceutical compositions of the present technology can be direct compression tablets or non-direct compression tablets. In at least one exemplary embodiment, a dosage form of the present technology can be made by wet granulation, and dry granulation (e.g., slugging or roller compaction). The method of preparation and type of excipients are selected to give the tablet formulation desired physical characteristics that allow for the rapid compression of the tablets. After compression, the tablets must have a number of additional attributes such as appearance, hardness, disintegrating ability, and an acceptable dissolution profile.

Choice of fillers and other excipients typically depends on the chemical and physical properties of the drug, behavior of the mixture during processing, and the properties of the final tablets. Adjustment of such parameters is understood to be within the general understanding of one skilled in the relevant art. Suitable fillers and excipients are described in more detail above.

The manufacture of a dosage form of the present technology can involve direct blend and compression, and wet and dry granulation methods, including slugging and roller compaction, for example.

Accordingly, and as described further below, a directly compressible pharmaceutical composition of the present technology can be designed following the teachings set forth herein that can deter one or more of a) parenteral abuse of a drug, b) inhalation abuse of a drug, and c) oral abuse of a drug.

Such compositions and dosage forms are formed according to the present technology as described herein. Steps for making the compositions or dosage forms include, for example, but not limited to, the step of providing at least one asalhydromorphone conjugate, or benzhydrocodone conjugate, or benzhydrocodone conjugate and acetaminophen combinatorial medicament as described above, an amount of a gel forming polymer having a desired molecular weight or viscosity as described above, a suitable amount of a disintegrant as described above, and/or providing a nasal tissue irritant, and/or providing an emetic in the amounts as described above.

Again, not wanting to be bound by any particular theory, it is believed that by controlling the molecular weight and/or viscosity of the gel forming polymer, and/or by controlling the amount of mucous membrane irritant and/or nasal tissue irritant such that nasal tissue irritation occurs if the composition is inhaled (e.g. snorting), and/or by controlling the amount of emetic such that emesis ensues if more than a prescribed amount of the active pharmaceutical ingredient is consumed, a therapeutic composition suitable for use to deter drug abuse can be formed. The compositions according to the presently described and claimed technology are believed to deter abuse of the opioid analgesic by (1) forming a viscous substance upon contact with a solvent such that the substance and analgesic cannot be easily drawn into a syringe and/or (2) by inducing mucous membrane irritation and/or nasal tissue irritation if the composition is inhaled, and/or (3) by inducing emesis if more than a prescribed amount of the analgesic is consumed.

It should be appreciated by those skilled in the art that the presently described and claimed technology can be used to manufacture immediate release, formulations.

For example, embodiments of the present technology may be prepared via melt techniques. In certain exemplary embodiments, the conjugate may be combined with one or more polymers of the present technology and optionally other ingredients to form a homogenous mixture and then the mixture can be subjected to a temperature for a duration sufficient to melt at least a portion of the polymer.

Immediate release matrices can also be prepared via melt-granulation or melt-extrusion techniques. In some embodiments, melt-granulation techniques involve melting a normally solid material and incorporating a powdered drug therein. In some embodiments, a homogenous mixture may be heated to a temperature sufficient to at least soften the mixture sufficiently to extrude the same.

### Pharmaceutical Kits

The present technology also provides pharmaceutical kits containing a specific amount of the individual doses in a package containing a composition of the present technology. The kit can further include instructions for use of the kit. The specified amount of individual doses may contain from about 1 to about 100 individual dosages, alternatively from about 1 to about 60 individual dosages, alternatively from about 10 to about 30 individual dosages, including, about 1, about 2, about 5, about 10, about 12, about 15, about 18, about 20, about 25, about 30, about 35, about 40, about 42, about 45, about 50, about 55, about 60, about 70, about 80, about 100, and include any additional increments thereof, for example, 1, 2, 5, 10 and multiplied factors thereof, (e.g., ×1, ×2, ×2.5, ×5, ×10, ×100, etc).

The following exemplary compositions illustrate different embodiments of the present technology.

### Abuse-Deterrent Composition 1

An abuse-deterrent composition may be formulated and may contain the following components:
i. at least one conjugate in an amount from 5 mg to 20 mg of hydrocodone conjugated to at least one ligand to provide abuse deterrence;
ii. a sufficient amount of at least one of polyethylene oxide having an average molecular weight of 900,000 to 7,000,000, hydroxypropyl methyl cellulose having a molecular weight ranging from about 10,000 to about 1,500,000, and/or a specific gravity of from 1.19 to 1.31, and/or an average specific gravity of 1.26, and/or a viscosity of 3600 to 5600, or carbomer having a molecular weight from 700,000 to 4,000,000,000, and/or a viscosity from 4000 cps to 39,400 cps, as a gel forming polymer;
iii. at least one disintegrant in an amount sufficient to cause the composition to exhibit an immediate release profile; and
iv. at least one surfactant in an amount of 1 percent to 20 percent by weight on a solid basis of the total composition.
   The hydrocodone can be conjugated to a benzoic acid ligand to form a benzoate-hydrocodone (benzhydrocodone) conjugate.

### Abuse-Deterrent Composition 2

An abuse-deterrent unit dose composition may be formulated and may contain the following components:
i. at least one conjugate combinatorial composition containing from 3 mg to 7 mg of hydrocodone conjugated to a benzoic acid ligand which can form a benzoate-hydrocodone (benzhydrocodone) conjugate, which is further combined with acetaminophen (in an amount of 300 mg to 325 mg) or a non-steroidal anti-inflammatory drug (NSAID) (in an amount of 25 mg to 1200 mg);
ii. a sufficient amount of at least one of polyethylene oxide having an average molecular weight of 900,000 to 7,000,000, hydroxypropyl methyl cellulose having a molecular weight ranging from about 10,000 to about 1,500,000, and/or a specific gravity of from 1.19 to 1.31, and/or an average specific gravity of 1.26, and/or a viscosity of 3600 to 5600, or carbomer having a molecular weight from 700,000 to 4,000,000,000, and/or a viscosity from 4000 cps to 39,400 cps, as a gel forming polymer;
iii. at least one disintegrant in an amount sufficient to cause the composition to exhibit an immediate release profile; and
iv. at least one surfactant in an amount of 1 percent to 20 percent by weight on a solid basis of the total composition.

### Abuse-Deterrent Composition 3

An abuse-deterrent composition may be formulated and may contain the following components:
i. at least one conjugate in an amount from 1 mg to 8 mg of hydromorphone conjugated to at least one ligand to provide abuse deterrence;
ii. a sufficient amount of at least one of polyethylene oxide having an average molecular weight of 900,000 to 7,000,000, hydroxypropyl methyl cellulose having a molecular weight ranging from about 10,000 to about 1,500,000, and/or a specific gravity of from 1.19 to 1.31, and/or an average specific gravity of 1.26, and/or a viscosity of 3600 to 5600, or carbomer having a molecular weight from 700,000 to 4,000,000,000, and/or a viscosity from 4000 cps to 39,400 cps, as a gel forming polymer;
iii. at least one disintegrant in an amount sufficient to cause the composition to exhibit an immediate release profile; and
iv. at least one surfactant in an amount of 1 percent to 20 percent by weight on a solid basis of the total composition.

The hydromorphone can be conjugated to aspirin (acetylsalicylate) ligands to form a 3,6-di-aspirin-hydromorphone (asalhydromorphone) conjugate.

### Abuse-Deterrent Composition 4

An abuse-deterrent composition may be formulated and may contain the following components:
i. at least one conjugate in an amount from 5 mg to 20 mg of hydrocodone conjugated to at least one ligand to provide abuse deterrence;
ii. a sufficient amount of at least one of polyethylene oxide having an average molecular weight of 900,000 to 7,000,000, hydroxypropyl methyl cellulose having a molecular weight ranging from about 10,000 to about 1,500,000, and/or a specific gravity of from 1.19 to 1.31, and/or an average specific gravity of 1.26, and/or a viscosity of 3600 to 5600, or carbomer having a molecular weight from 700,000 to 4,000,000,000, and/or a viscosity from 4000 cps to 39,400 cps, as a gel forming polymer;
iii. crospovidone having a particle size distribution of about 400 microns or less and a density of about 1.22 g/mL as a disintegrant, in an amount sufficient to cause the composition to exhibit an immediate release profile; and
iv. at least one of sodium lauryl sulfate, poloxamer, sorbitan monoesters, glyceryl monooleates, or a combination thereof in an amount of 1 percent to 20 percent by weight on a solid basis of the total composition.

The hydrocodone can be conjugated to a benzoic acid ligand to form a benzoate-hydrocodone (benzhydrocodone) conjugate.

Abuse-Deterrent Composition 5

An abuse-deterrent composition may be formulated and may contain the following components:
i. at least one conjugate in an amount from 5 mg to 20 mg of hydrocodone conjugated to at least one ligand to provide abuse deterrence;
ii. a sufficient amount of at least one of polyethylene oxide having an average molecular weight of 900,000 to 7,000,000, hydroxypropyl methyl cellulose having a molecular weight ranging from about 10,000 to about 1,500,000, and/or a specific gravity of from 1.19 to 1.31, and/or an average specific gravity of 1.26, and/or a viscosity of 3600 to 5600, or carbomer having a molecular weight from 700,000 to 4,000,000,000, and/or a viscosity from 4000 cps to 39,400 cps, as a gel forming polymer;
iii. crospovidone having a maximum particle size of about 400 microns and a density of about 1.22 g/mL as a disintegrant, in an amount sufficient to cause the composition to exhibit an immediate release profile; and
iv. at least one of sodium lauryl sulfate, poloxamer, sorbitan monoesters, glyceryl monooleates, or a combination thereof in an amount of 1 percent to 20 percent by weight on a solid basis of the total composition.

The hydrocodone can be conjugated to a benzoic acid ligand to form a benzoate-hydrocodone (benzhydrocodone) conjugate.

### Abuse-Deterrent Composition 6

An abuse-deterrent unit dose composition may be formulated and may contain the following components:
i. at least one conjugate combinatorial composition containing from 3 mg to 7 mg of hydrocodone conjugated to a benzoic acid ligand which can form a benzoate-hydrocodone (benzhydrocodone) conjugate, which is further combined with acetaminophen (in an amount of 300 mg to 325 mg) or a non-steroidal anti-inflammatory drug (NSAID) (in an amount of 25 mg to 1200 mg);
ii. a sufficient amount of at least one of polyethylene oxide having an average molecular weight of 900,000 to 7,000,000, hydroxypropyl methyl cellulose having a molecular weight ranging from about 10,000 to about 1,500,000, and/or a specific gravity of from 1.19 to 1.31, and/or an average specific gravity of 1.26, and/or a viscosity of 3600 to 5600, or carbomer having a molecular weight from 700,000 to 4,000,000,000, and/or a viscosity from 4000 cps to 39,400 cps, as a gel forming polymer;
iii. crospovidone having a particle size distribution of about 400 microns or less and a density of about 1.22 g/mL as a disintegrant, in an amount sufficient to cause the composition to exhibit an immediate release profile; and
iv. at least one of sodium lauryl sulfate, poloxamer, sorbitan monoesters, glyceryl monooleates, or a combination thereof in an amount of 1 percent to 20 percent by weight on a solid basis of the total composition.

### Abuse-Deterrent Composition 7

An abuse-deterrent unit dose composition may be formulated and may contain the following components:
i. at least one conjugate combinatorial composition containing from 3 mg to 7 mg of hydrocodone conjugated to a benzoic acid ligand which can form a benzoate-hydrocodone (benzhydrocodone) conjugate, which is further combined with acetaminophen (in an amount of 300 mg to 325 mg) or a non-steroidal anti-inflammatory drug (NSAID) (in an amount of 25 mg to 1200 mg);
ii. a sufficient amount of at least one of polyethylene oxide having an average molecular weight of 900,000 to 7,000,000, hydroxypropyl methyl cellulose having a molecular weight ranging from about 10,000 to about 1,500,000, and/or a specific gravity of from 1.19 to 1.31, and/or an average specific gravity of 1.26, and/or a viscosity of 3600 to 5600, or carbomer having a molecular weight from 700,000 to 4,000,000,000, and/or a viscosity from 4000 cps to 39,400 cps, as a gel forming polymer;
iii. crospovidone having a maximum particle size of about 400 microns and a density of about 1.22 g/mL as a disintegrant, in an amount sufficient to cause the composition to exhibit an immediate release profile; and
iv. at least one of sodium lauryl sulfate, poloxamer, sorbitan monoesters, glyceryl monooleates, or a combination thereof in an amount of 1 percent to 20 percent by weight on a solid basis of the total composition.

### Abuse-Deterrent Composition 8

An abuse-deterrent unit dose composition may be formulated and may contain the following components:
i at least one conjugate combinatorial composition containing from 3 mg to 7 mg of hydrocodone conjugated to a benzoic acid ligand which can form a hydrocodone-benzoate (benzhydrocodone) conjugate, which is further combined with acetaminophen (in an amount of 300 mgs to 325 mgs) or a non-steroidal anti-inflammatory drug (NSAID) (in an amount of 25 mgs to 1200 mgs);
ii a sufficient amount of at least one of polyethylene oxide having an average molecular weight of 900,000 to 7,000,000, hydroxypropyl methyl cellulose having a molecular weight ranging from about 10,000 to about 1,500,000, and/or a specific gravity of from 1.19 to 1.31, and/or an average specific gravity of 1.26, and/or a viscosity of 3600 to 5600, or carbomer having a molecular weight from 700,000 to 4,000,000,000, and/or a viscosity from 4000 cps to 39,400 cps, as a gel forming polymer;
iii crospovidone having a maximum particle size of about 400 microns and a density of about 1.22 g/mL as a disintegrant, in an amount sufficient to cause the composition to exhibit an immediate release profile; and
iv at least one of sodium lauryl sulfate, poloxamer, sorbitan monoesters, glyceryl monooleates, or a combination thereof in an amount of 1 percent to 20 percent by weight on a solid basis of the total composition; and:
v at least one binder, wherein the binder comprises povidone.

### Abuse-Deterrent Composition 9

An abuse-deterrent unit dose composition may be formulated and may contain the following components:
i. at least one conjugate combinatorial composition containing from 3 mg to 7 mg of hydrocodone conjugated to a benzoic acid ligand which can form a hydrocodone-benzoate (benzhydrocodone) conjugate, which is further combined with acetaminophen (in an amount of 100 mgs to 325 mgs);
ii. a sufficient amount of at least one of polyethylene oxide having an average molecular weight of 900,000 to 7,000,000, hydroxypropyl as a gel forming polymer;
iii. crospovidone having a maximum particle size of about 400 microns and a density of about 1.22 g/ml as a disintegrant, in an amount sufficient to cause the composition to exhibit an immediate release profile; and
iv. at least one surfactant, wherein the surfactant is sodium lauryl sulfate, in an amount of 1 percent to 20 percent by weight on a solid basis of the total composition; and:
v. at least one binder.

### Abuse-Deterrent Composition 10

An abuse-deterrent composition may be formulated and may contain the following components:
i. at least one conjugate in an amount from 1 mg to 8 mg of hydromorphone conjugated to at least one ligand to provide abuse deterrence;
ii. a sufficient amount of at least one of polyethylene oxide having an average molecular weight of 900,000 to 7,000,000, hydroxypropyl methyl cellulose having a molecular weight ranging from about 10,000 to about 1,500,000, and/or a specific gravity of from 1.19 to 1.31, and/or an average specific gravity of 1.26, and/or a viscosity of 3600 to 5600, or carbomer having a molecular weight from 700,000 to 4,000,000,000, and/or a viscosity from 4000 cps to 39,400 cps, as a gel forming polymer;
iii. crospovidone having a particle size distribution of about 400 microns or less and a density of about 1.22 g/mL as a disintegrant, in an amount sufficient to cause the composition to exhibit an immediate release profile; and
iv. at least one of sodium lauryl sulfate, poloxamer, sorbitan monoesters, glyceryl monooleates, or a combination thereof in an amount of 1 percent to 20 percent by weight on a solid basis of the total composition.

The hydromorphone can be conjugated to aspirin (acetylsalicylate) ligands to form a 3,6-di-aspirin-hydromorphone (asalhydromorphone) conjugate.

### Abuse-Deterrent Composition 11

An abuse-deterrent composition may be formulated and may contain the following components:
i. at least one conjugate in an amount from 1 mg to 8 mg of hydromorphone conjugated to at least one ligand to provide abuse deterrence;
ii. a sufficient amount of at least one of polyethylene oxide having an average molecular weight of 900,000 to 7,000,000, hydroxypropyl methyl cellulose having a molecular weight ranging from about 10,000 to about 1,500,000, and/or a specific gravity of from 1.19 to 1.31, and/or an average specific gravity of 1.26, and/or a viscosity of 3600 to 5600, or carbomer having a molecular weight from 700,000 to 4,000,000,000, and/or a viscosity from 4000 cps to 39,400 cps, as a gel forming polymer;
iii. crospovidone having a maximum particle size of about 400 microns and a density of about 1.22 g/mL as a disintegrant, in an amount sufficient to cause the composition to exhibit an immediate release profile; and
iv. at least one of sodium lauryl sulfate, poloxamer, sorbitan monoesters, glyceryl monooleates, or a combination thereof in an amount of 1 percent to 20 percent by weight on a solid basis of the total composition.

The hydromorphone can be conjugated to aspirin (acetylsalicylate) ligands to form a 3,6-di-aspirin-hydromorphone (asalhydromorphone) conjugate.

### Abuse-Deterrent Composition 12

An abuse-deterrent composition may be formulated and may contain the following components:
ii. at least one conjugate in an amount from 5 mg to 20 mg of hydrocodone conjugated to at least one ligand to provide abuse deterrence;
iii. a sufficient amount of at least one of polyethylene oxide having an average molecular weight of 900,000 to 7,000,000, hydroxypropyl methyl cellulose having a molecular weight ranging from about 10,000 to about 1,500,000, and/or a specific gravity of from 1.19 to 1.31, and/or an average specific gravity of 1.26, and/or a viscosity of 3600 to 5600, or carbomer having a molecular weight from 700,000 to 4,000,000,000, and/or a viscosity from 4000 cps to 39,400 cps, as a gel forming polymer;
iv. sodium starch glycolate having a particle size of about 104 microns and a density of about 0.756 g/mL, or croscarmellose sodium having a particle size of about 37 microns to about 73.7 microns and a density of about 0.529 g/mL; as a disintegrant, in an amount sufficient to cause the composition to exhibit an immediate release profile; and
v. at least one of sodium lauryl sulfate, poloxamer, sorbitan monoesters, glyceryl monooleates, or a combination thereof in an amount of 1 percent to 20 percent by weight on a solid basis of the total composition.

The hydrocodone can be conjugated to a benzoic acid ligand to form a benzoate-hydrocodone (benzhydrocodone) conjugate.

### Abuse-Deterrent Composition 13

An abuse-deterrent unit dose composition may be formulated and may contain the following components:
i. at least one conjugate combinatorial composition containing from 3 mg to 7 mg of hydrocodone conjugated to a benzoic acid ligand which can form a benzoate-hydrocodone (benzhydrocodone) conjugate which is further combined with acetaminophen (in an amount of 300 mg to 325 mg) or a non-steroidal anti-inflammatory drug (NSAID) (in an amount of 25 mg to 1200 mg);
ii. a sufficient amount of at least one of polyethylene oxide having an average molecular weight of 900,000 to 7,000,000, hydroxypropyl methyl cellulose having a molecular weight ranging from about 10,000 to about 1,500,000, and/or a specific gravity of from 1.19 to 1.31, and/or an average specific gravity of 1.26, and/or a viscosity of 3600 to 5600, or carbomer having a molecular weight from 700,000 to 4,000,000,000, and/or a viscosity from 4000 cps to 39,400 cps, as a gel forming polymer;
iii. sodium starch glycolate having a particle size of about 104 microns and a density of about 0.756 g/mL, or croscarmellose sodium having a particle size of about 37 microns to about 73.7 microns and a density of about 0.529 g/mL; as a disintegrant, in an amount sufficient to cause the composition to exhibit an immediate release profile; and
iv. at least one of sodium lauryl sulfate, poloxamer, sorbitan monoesters, glyceryl monooleates, or a combination thereof in an amount of 1 percent to 20 percent by weight on a solid basis of the total composition.
Abuse-Deterrent Composition 14

An abuse-deterrent composition may be formulated and may contain the following components:
i. at least one conjugate in an amount from 1 mg to 8 mg of hydromorphone conjugated to at least one ligand to provide abuse deterrence;
ii. a sufficient amount of at least one of polyethylene oxide having an average molecular weight of 900,000 to 7,000,000, hydroxypropyl methyl cellulose having a molecular weight ranging from about 10,000 to about 1,500,000, and/or a specific gravity of from 1.19 to 1.31, and/or an average specific gravity of 1.26, and/or a viscosity of 3600 to 5600, or carbomer having a molecular weight from 700,000 to 4,000,000,000, and/or a viscosity from 4000 cps to 39,400 cps, as a gel forming polymer;
iii. sodium starch glycolate having a particle size of about 104 microns and a density of about 0.756 g/mL, or croscarmellose sodium having a particle size of about 37 microns to about 73.7 microns and a density of about 0.529 g/mL; as a disintegrant, in an amount sufficient to cause the composition to exhibit an immediate release profile; and
iv. at least one of sodium lauryl sulfate, poloxamer, sorbitan monoesters, glyceryl monooleates, or a combination thereof in an amount of 1 percent to 20 percent by weight on a solid basis of the total composition.

The hydromorphone can be conjugated to aspirin (acetylsalicylate) ligands to form a 3,6-di-aspirin-hydromorphone (asalhydromorphone) conjugate.

### Abuse-Deterrent Composition 15

An abuse-deterrent composition may be formulated and may contain the following components:
i. at least one conjugate in an amount from 5 mg to 20 mg of hydrocodone conjugated to at least one ligand to provide abuse deterrence;
ii. a sufficient amount of at least one of polyethylene oxide having an average molecular weight of 900,000 to 7,000,000, hydroxypropyl methyl cellulose having a molecular weight ranging from about 10,000 to about 1,500,000, and/or a specific gravity of from 1.19 to 1.31, and/or an average specific gravity of 1.26, and/or a viscosity of 3600 to 5600, or carbomer having a molecular weight from 700,000 to 4,000,000,000, and/or a viscosity from 4000 cps to 39,400 cps, as a gel forming polymer;
iii. starch having a particle size of 2 microns to 32 microns and a density of 0.462 g/mL in combination with at least one of crospovidone having a particle size distribution of about 400 microns or less and a density of about 1.22 g/mL, sodium starch glycolate having a particle size of about 104 microns and a density of about 0.756 g/mL, or croscarmellose sodium having a particle size of about 37 microns to about 73.7 microns and a density of about 0.529 g/mL; as a disintegrant, in an amount sufficient to cause the composition to exhibit an immediate release profile; and
iv. at least one of sodium lauryl sulfate, poloxamer, sorbitan monoesters, glyceryl monooleates, or a combination thereof in an amount of 1 percent to 20 percent by weight on a solid basis of the total composition.

The hydrocodone can be conjugated to a benzoic acid ligand to form a benzoate-hydrocodone (benzhydrocodone) conjugate.

### Abuse-Deterrent Composition 16

An abuse-deterrent unit dose composition may be formulated and may contain the following components:
i. at least one conjugate combinatorial composition containing from 3 mg to 7 mg of hydrocodone conjugated to a benzoic acid ligand which can form a benzoate-hydrocodone (benzhydrocodone) conjugate which is further combined with acetaminophen (in an amount of 300 mg to 325 mg) or a non-steroidal anti-inflammatory drug (NSAID) (in an amount of 25 mg to 1200 mg);
ii. a sufficient amount of at least one of polyethylene oxide having an average molecular weight of 900,000 to 7,000,000, hydroxypropyl methyl cellulose having a molecular weight ranging from about 10,000 to about 1,500,000, and/or a specific gravity of from 1.19 to 1.31, and/or an average specific gravity of 1.26, and/or a viscosity of 3600 to 5600, or carbomer having a molecular weight from 700,000 to 4,000,000,000, and/or a viscosity from 4000 cps to 39,400 cps, as a gel forming polymer;
iii. starch having a particle size of 2 microns to 32 microns and a density of 0.462 g/mL in combination with at least one of crospovidone having a particle size distribution of about 400 microns or less and a density of about 1.22 g/mL, sodium starch glycolate having a particle size of about 104 microns and a density of about 0.756 g/mL, or croscarmellose sodium having a particle size of about 37 microns to about 73.7 microns and a density of about 0.529 g/mL; as a disintegrant, in an amount sufficient to cause the composition to exhibit an immediate release profile; and
iv. at least one of sodium lauryl sulfate, poloxamer, sorbitan monoesters, glyceryl monooleates, or a combination thereof in an amount of 1 percent to 20 percent by weight on a solid basis of the total composition.

### Abuse-Deterrent Composition 17

An abuse-deterrent composition may be formulated and may contain the following components:
i. at least one conjugate in an amount from 1 mg to 8 mg of hydromorphone conjugated to at least one ligand to provide abuse deterrence;
ii. a sufficient amount of at least one of polyethylene oxide having an average molecular weight of 900,000 to 7,000,000, hydroxypropyl methyl cellulose having a molecular weight ranging from about 10,000 to about 1,500,000, and/or a specific gravity of from 1.19 to 1.31, and/or an average specific gravity of 1.26, and/or a viscosity of 3600 to 5600, or carbomer having a molecular weight from 700,000 to 4,000,000,000, and/or a viscosity from 4000 cps to 39,400 cps, as a gel forming polymer;
iii. starch having a particle size of 2 microns to 32 microns and a density of 0.462 g/mL in combination with at least one of crospovidone having a particle size distribution of about 400 microns or less and a density of about 1.22 g/mL, sodium starch glycolate having a particle size of about 104 microns and a density of about 0.756 g/mL, or croscarmellose sodium having a particle size of about 37 microns to about 73.7 microns and a density of about 0.529 g/mL; as a disintegrant, in an amount sufficient to cause the composition to exhibit an immediate release profile; and
iv. at least one of sodium lauryl sulfate, poloxamer, sorbitan monoesters, glyceryl monooleates, or a combination thereof in an amount of 1 percent to 20 percent by weight on a solid basis of the total composition.

The hydromorphone can be conjugated to aspirin (acetylsalicylate) ligands to form a 3,6-di-aspirin-hydromorphone (asalhydromorphone) conjugate.

Certain aspects of the present invention may be better understood as illustrated by the following examples, which are meant by way of illustration and not limitation. Further, such Examples illustrate various aspects and embodiments of the present technology, including but not limited to, the conjugation technology aspects of the presently described and claimed technology.

### ILLUSTRATIVE EXAMPLES

### Example 1: Chemical Stability of Benzoate and Heteroaryl Carboxylate Conjugates of Hydrocodone

Exemplary conjugates of hydrocodone of the present technology and control test conjugates not of the present technology were tested for chemical stability under conditions similar to what a potential drug abuser may use to “extract” the active portion of the molecule, for example dissolved in water, hydrochloric acid or sodium bicarbonate either at ambient temperature or 100° C. The conjugates were placed in a solution of water at either ambient temperature (about 20° C.) or in an oil bath at 100° C. for one hour and the amount of the conjugate that was hydrolyzed under these conditions was measured. Table 1 demonstrates the results, showing that the conjugates did not release hydrocodone at ambient temperature or when heated in water to 100° C. for one hour.

TABLE 1

| | water^{a} | |
|---|---|---|
| Compound | ambient | 100° C. |
| 4-OH-Bz-HC | 0% | 0% |
| 2-Abz-HC | 0% | 0% |
| 4-MeO-Bz-HC | 0% | 0% |

Further, samples of conjugates of hydrocodone of the present technology were tested and compared with samples of other conjugates not of the present technology of hydrocodone (Adipate-HC) for their hydrolysis to hydrocodone after dilution in 1 N hydrochloric acid (HCl) for 1 hour at ambient temperature (˜20° C.) or in an oil bath at 100° C. The percentages indicate how much of the initial amount of conjugate was hydrolyzed under these conditions. The results are shown in Table 2.

TABLE 2

| | %-release in 1 N HCl^{a} | |
|---|---|---|
| Compound | ambient | 100° C. |
| 4-OH-Bz-HC | 0% | 30% |
| 2-Abz-HC | 0% | 16% |
| 3-OH-4-MeO-Bz-HC | 0% | 35% |
| 2-OH-Bz-HC | 3% | 27% |
| Adipate-HC | 13% | 100% |

Samples of each conjugate were dissolved in a solution of 5% NaHCO₃ for one hour at either ambient temperature (˜20° C.) or in an oil bath at 100° C. The percentages indicate how much of the initial amount of conjugate was hydrolyzed under these conditions as shown in Table 3 for the conjugates of the present technology and comparison conjugates not of the present technology (Tyr-Tyr-Phe-Phe-Ile-Hydrocodone (YYFFI-HC) or Adipiate-HC).

TABLE 3

| | %-release in 5% NaHCO₃^{a} | |
|---|---|---|
| Compound | ambient | 100° C. |
| 4-OH-Bz-HC | 1% | 23% |
| 3-OH-4-MeO-Bz-HC | 0% | 36% |
| YYFFI-HC | 0% | 70% |
| Adipate-HC | 3% | 100% |

### Example 2: Oral PK Profiles of Conjugated Hydrocodone of the Present Technology

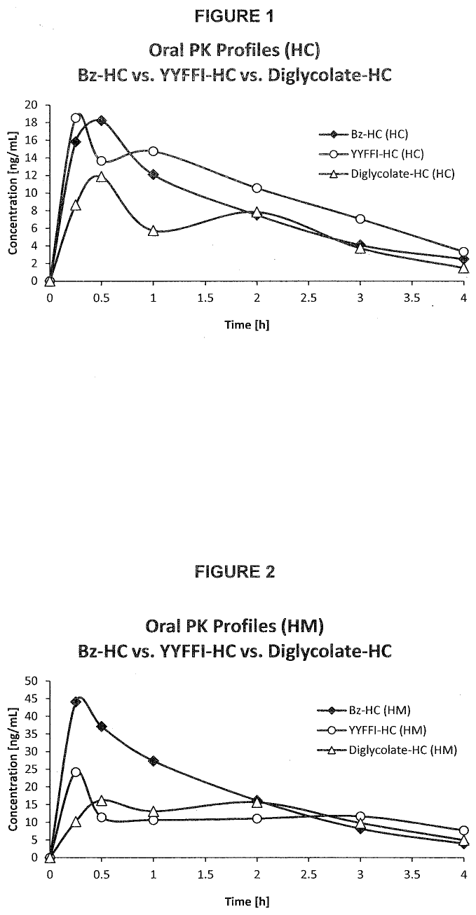

Oral PK curves were determined for benzhydrocodone (Bz-HC), a conjugate of the present technology, as compared to two conjugates not within the scope of the present technology: YYFFI-HC and Diglycolate-HC. Rats were orally administered an amount of the conjugate equivalent to 2 mg/kg of freebase hydrocodone and the plasma concentrations of released hydrocodone and of the active metabolite hydromorphone were measured over time by LC-MS/MS. As shown in FIG. 1, the oral PK curves for released hydrocodone were somewhat similar for Bz-HC and YYFFI-HC, but hydrocodone plasma concentrations produced by Bz-HC were mostly significantly higher than hydrocodone concentrations generated by Diglycolate-HC (AUC and Cₘₐₓ for Bz-HC were approximately 40% and 50% higher, respectively). Additionally, Bz-HC created higher plasma concentrations of the more potent active metabolite hydromorphone (FIG. 2) than both, YYFFI-HC (AUC and Cₘₐₓ for hydromorphone released from Bz-HC were approximately 60% and 80% higher, respectively) and Diglycolate-HC (AUC and Cₘₐₓ for hydromorphone released from Bz-HC were approximately 55% and 180% higher, respectively). This suggests that all three compounds undergo a different metabolic pathway and that Bz-HC would have pain relieving effects potentially greater than either example.

### Example 3: Intranasal PK Profile of Conjugates of Hydrocodone

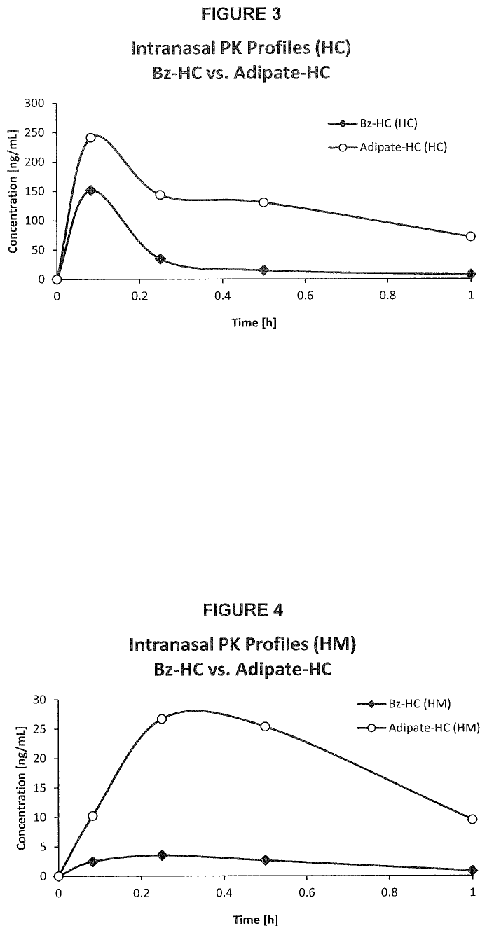

Conjugates of hydrocodone of the present technology were tested for abuse resistance capabilities by examining the efficiency of a hydrolysis when administered via routes other than oral. Rats were intranasally treated with conjugate in an amount equivalent to 2 mg/kg of hydrocodone freebase and the concentration of released hydrocodone and of the active metabolite hydromorphone in the plasma of the rat were measured over time by LC-MS/MS. Hydrocodone plasma concentrations were significantly lower for Bz-HC (AUC and Cₘₐₓ for hydromorphone released from Adipate-HC were approximately 280% and 60% higher, respectively) as shown in FIG. 3. Moreover, Bz-HC produced very low plasma concentration of hydromorphone when compared to Adipate-HC (AUC and Cₘₐₓ for hydromorphone released from Adipate-HC were approximately 750% and 660% higher, respectively) as shown in FIG. 4.

Conjugates of the present technology provide hydrocodone and hydromorphone plasma concentrations that are significantly lower than respective plasma concentration for unbound Hydrocodone.BT when administered intranasally.

### Example 4: Exemplary Intravenous PK Profiles of Conjugates of the Present Technology

The conjugates of hydrocodone of the present technology are hydrophobic, for example, Bz-HC, Nicotinate-HC, 4-MeO-Bz-HC, Piperonylate-HC, 4-OH-Bz-HC, Salicylate-HC, 3-OH-4-MeO-Bz-HC, 3-OH-Bz-HC and Gallate-HC. Therefore, these compounds cannot be administered intravenously at oral equivalent doses because they do not dissolve in a practical amount of water since injectable compounds must be completely in solution, because any solid particle may cause an embolism. The amount of water necessary to dissolve a desirable amount of conjugate would make an injection unfeasible and thus the present compositions and conjugates have anti-abuse potential as opposed to other hydrocodone conjugates that are water soluble, such as Adipate-HC and Diglycolate-HC which can be administered intravenously at oral equivalent doses.

### Example 5: Comparison of Oral PK Profiles of Conjugates of Hydrocodone

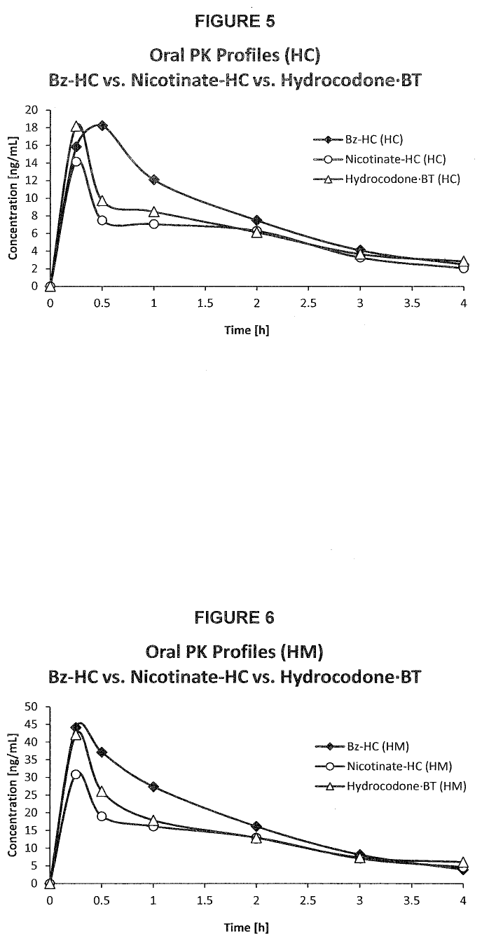

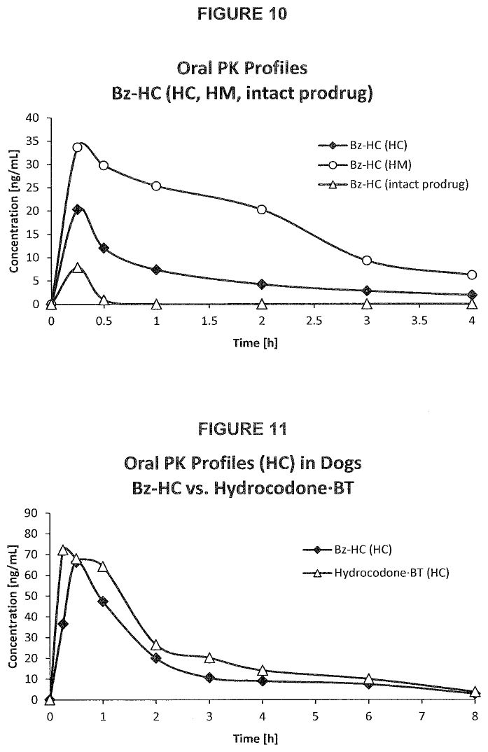

The plasma concentrations of hydrocodone released from Bz-HC and Nicotinate-HC were compared to plasma concentrations of hydrocodone generated by unconjugated Hydrocodone.BT after oral administration to rats. Rats were treated with conjugated or unconjugated drug in an amount equivalent to 2 mg/kg of hydrocodone freebase and the plasma concentration of hydrocodone or hydromorphone was measured by LC-MS/MS as demonstrated in FIGS. 5 and 10 respectively. The oral plasma concentration of hydrocodone released from Bz-HC increased similarly to the hydrocodone plasma concentrations observed with Hydrocodone.BT, until it reached Cₘₐₓ (Cₘₐₓ was approximately equal for both compounds). After Tₘₐₓ, the hydrocodone plasma concentration for Bz-HC decreased in a slower and more controlled fashion than for unconjugated Hydrocodone.BT (FIG. 5 and FIG. 6). Bz-HC had a higher AUC (AUC was approximately 25% higher, FIG. 5) when compared to Hydrocodone.BT and similar results were observed for the plasma concentrations of the active metabolite hydromorphone (FIG. 6).

Nicotinate-HC, produced hydrocodone and hydromorphone plasma concentrations that were below the respective concentrations found for unconjugated Hydrocodone.BT. The corresponding AUC values, however, were within the range of bioequivalence for the same dose (based on hydrocodone freebase).

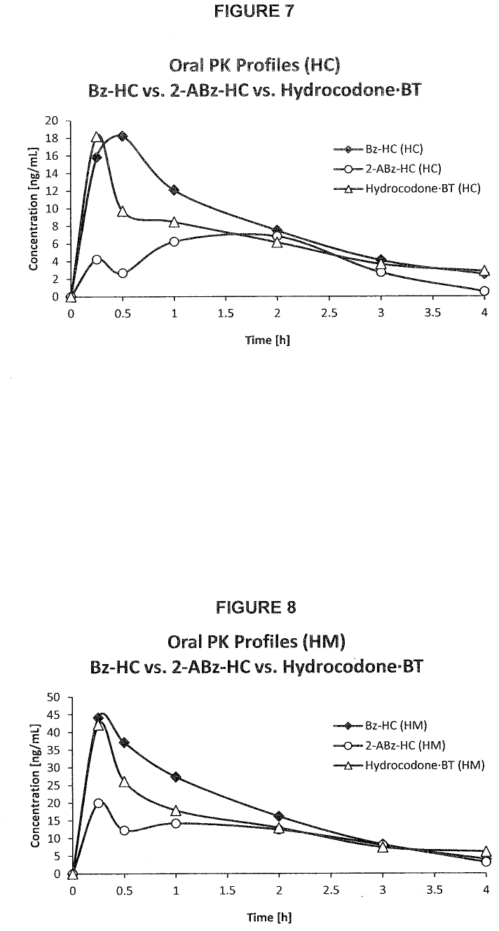

2-ABz-HC demonstrated a different release profile after oral administration to rats than Bz-HC or the unconjugated drug Hydrocodone.BT. Rats were treated with an amount equivalent to 2 mg/kg of hydrocodone freebase and the plasma concentration of hydrocodone or hydromorphone was measured by LC-MS/MS over time as shown in FIG. 7 or FIG. 8 respectively. 2-ABz-HC released hydrocodone very slowly indicated by a gradual increase of plasma concentration followed by an attenuated decrease (FIG. 7). This resulted in a flattened PK curve when compared with Hydrocodone.BT (Tₘₐₓ for 2-ABz-HC was approximately four times longer, AUC and Cₘₐₓ were approximately 35% and 60% lower, respectively). Overall, the PK curve of hydromorphone was also flatter for 2-ABz-HC than for Hydrocodone.BT (FIG. 8) but did show a small initial spike (AUC and Cₘₐₓ for 2-ABz-HC were approximately 25% and 50% lower, respectively).

### Example 6: Determination of Variation in Plasma Concentrations of Benzhydrocodone

To determine the variability of the plasma concentration of hydrocodone (HC) and hydromorphone (HM), the coefficient of variation (CV) was calculated for individual animals that were dosed with an amount equivalent to 2 mg/kg of hydrocodone freebase of benzhydrocodone or the unconjugated hydrocodone bitartrate (BT) and the plasma concentrations of hydrocodone and hydromorphone were measured by LC-MS/MS over time. The CV was calculated by dividing the standard deviation of plasma concentrations in individual animals by the mean plasma concentrations of all dosed animals for a given time point. The “average CV” is the mean CV for all time points, as shown in Table 4.

TABLE 4

| | Average CV^{a} | |
|---|---|---|
| Compound | HC | HM |
| Bz-HC | 46 | 41 |
| Hydrocodone•BT | 75 | 64 |

The lower average CV for Bz-HC indicates that this conjugate has lower relative variability in plasma concentrations of hydrocodone and hydromorphone across all dosed animals and time points than the unconjugated drug, hydrocodone bitartrate.

### Example 7: Synthesis of Conjugates of Hydrocodone

### Synthesis of Benzhydrocodone Freebase

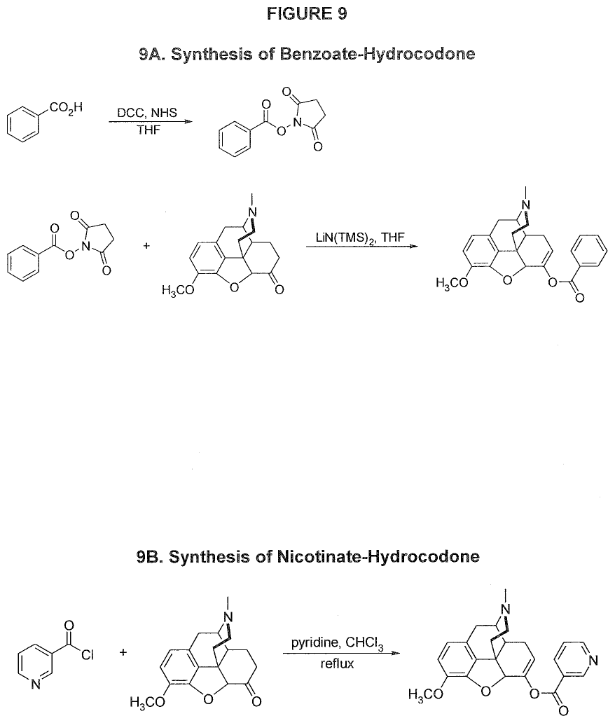

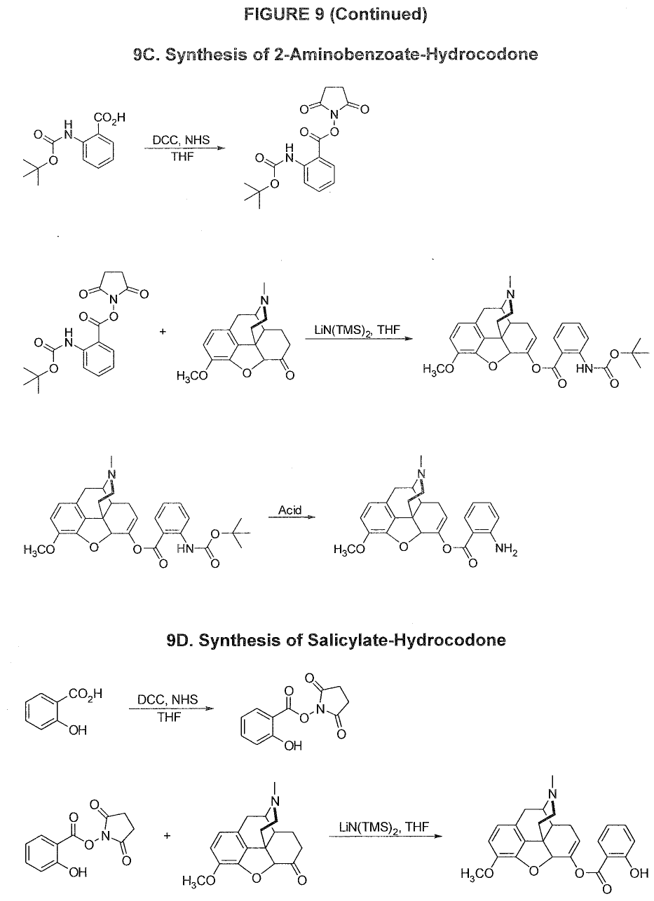

To a solution of hydrocodone freebase (0.596 g, 1.99 mmol) in tetrahydrofuran (25 mL) was added 1 M LiN(SiMe₃)₂ in tetrahydrofuran (5.98 mL). The resulting orange suspension was stirred at ambient temperatures for 30 min. after which benzoate-succinic ester (1.25 g, 5.98 mmol) was added. The resulting mixture was stirred overnight at ambient temperatures and was quenched after 18 h by the addition of 100 mL saturated ammonium chloride solution which was allowed to stir for another 2 h. Ethyl acetate (100 mL) was added to the mixture and washed with saturated ammonium chloride solution (3×100 mL) and water (1×100 mL). Organic extracts were dried over anhydrous MgSO₄, solvent was removed and residue was taken up in 2-isopropanol (50 mL). Water was added until a solid formed. The resulting mixture was chilled, filtered and dried to obtain benzhydrocodone freebase (0.333 g, 0.826 mmol, 42% yield) as a dark brown solid. This synthesis is depicted in FIG. 9A.

### Synthesis of 2-Boc-Aminobenzoic Succinate

2-Boc-aminobenzoic acid (2.56 g, 10.8 mmol) and N-hydroxysuccinimide (1.37 g, 11.88 mmol) were dissolved in 25 mL of THF. DCC (2.45 g, 11.88 mmol) was added in one portion. The reaction was stirred overnight. The solid was filtered off and rinsed with acetone (2×10 mL). The filtrate was concentrated to dryness and dissolved in 100 mL of acetone. The resulting precipitate (DCU) was filtered off and the filtrate was concentrated to give a solid, which was collected and rinsed with methanol (3×4 mL) to yield 3.26 g (90%) of white product.

### Synthesis of 2-Boc-Aminobenzoic Acid Ester of Hydrocodone

To hydrocodone freebase (0.449 g, 1.5 mmol) dissolved in 20 mL of anhydrous THF was added a solution of LiHMDS in THF (1 M, 4.5 mL, 4.5 mmol) over 20 min. The mixture was stirred for 30 min. and 2-Boc-aminobenzoic succinate (1.50 g, 4.5 mmol) was added in one portion. The reaction was stirred for 4 hr and subsequently quenched with 100 mL of sat. NH₄Cl. The mixture was stirred for 1 hr. and extracted with 200 mL of ethyl acetate. The ethyl acetate layer was washed with sat. NaHCO₃ (2×80 mL) and 5% brine (80 mL), dried over anhydrous Na₂SO₄ and concentrated. The residue was purified by silica gel column chromatography (7% MeOH/CH₂Cl₂) to give 449 mg (58%) of an amorphous solid.

### Synthesis of 2-Aminobenzoic Acid Ester of Hydrocodone Dihydrochloride Salt

2-Boc-aminobenzoic acid ester of hydrocodone (259 mg, 0.5 mmol) was stirred in 4 mL of 4 N HCl/dioxane for 4 hr. The solvent was evaporated to dryness and to the residue was added 5 mL of ethyl acetate. The solid was collected and rinsed with ethyl acetate to give 207 mg (84%) of product.

### Synthesis of 2-MOM-salicylic succinate

2-MOM-salicylic acid (3.2 g, 17.6 mmol) and N-hydroxysuccinimide (2.23 g, 19.36 mmol) were dissolved in 40 mL of THF. DCC (3.99 g, 19.36 mmol) was added in one portion. The reaction was stirred overnight. The solid was filtered off and rinsed with acetone (2×10 mL). The filtrate was concentrated and the residue was recrystallized from 10 mL of methanol to give 2.60 g (53%) of a white solid.

### Synthesis of 2-MOM-Salicylic Acid Ester of Hydrocodone

To hydrocodone freebase (0.449 g, 1.5 mmol) dissolved in 20 mL of anhydrous THF was added a solution of LiHMDS in THF (1 M, 4.5 mL, 4.5 mmol) over 20 min. The mixture was stirred for 30 min. and 2-MOM-salicylic succinate (1.26 g, 4.5 mmol) was added in one portion. The reaction was stirred for 4 hr. and subsequently quenched with 100 mL of sat. NH₄Cl. The mixture was stirred for 1 hr. and extracted with 200 mL of ethyl acetate. The ethyl acetate layer was washed with sat. NaHCO₃ (2×80 mL) and 5% brine (80 mL), dried over anhydrous Na₂SO₄ and concentrated. The residue was purified by silica gel column chromatography (8% MeOH/CH₂Cl₂) to give 381 mg (58%) of a syrup.

### Synthesis of Salicylic Acid Ester of Hydrocodone Hydrochloride Salt

To 2-MOM-salicylic acid ester of hydrocodone (380 mg, 0.82 mmol) in 12 mL of methanol was added 0.5 mL of conc. HCl (12 N). The reaction was stirred for 6 hr. The solution was concentrated and residual water was removed by coevaporating with methanol (5×5 mL). The resulting residue was dissolved in 1 mL of methanol followed by 20 mL of ethyl acetate. The cloudy mixture was evaporated to about 4 mL. The resulting solid was collected and rinsed with ethyl acetate to yield 152 mg (41%) of product.

### Example 8: Oral PK Profiles of Conjugated Hydrocodone, Hydrocodone, and Hydromorphone in Rats

After oral administration of benzhydrocodone (Bz-HC) to rats, PK curves were determined for intact Bz-HC, hydrocodone, and the active metabolite hydromorphone. Rats were orally administered an amount of the conjugate equivalent to 2 mg/kg of freebase hydrocodone and the plasma concentrations of intact Bz-HC, released hydrocodone, and the active metabolite, hydromorphone, were measured over time by LC-MS/MS. As shown in FIG. 10, the exposure to intact Bz-HC conjugate was much lower than the exposure to hydrocodone or hydromorphone (the AUC for intact Bz-HC was approximately 10% and 3% of the AUC values for hydrocodone and hydromorphone, respectively).

### Example 9: Oral PK Profiles of Conjugated Hydrocodone, Hydrocodone, and Hydromorphone in Dogs

After oral administration of benzhydrocodone (Bz-HC) or Hydrocodone.BT to dogs, PK curves were determined for intact Bz-HC (Bz-HC arm only), hydrocodone, and the active metabolite hydromorphone. Dogs were orally administered an amount of Hydrocodone.BT or the conjugate equivalent to 2 mg/kg of freebase hydrocodone. The plasma concentrations of intact Bz-HC, released hydrocodone, and the active metabolite, hydromorphone, were measured over time by LC-MS/MS.

A comparison of plasma concentrations of hydrocodone released from Bz-HC and Hydrocodone.BT is shown in FIG. 11. Overall, the plasma concentrations of hydrocodone generated by both compounds were quite similar. The systemic exposure to hydrocodone was somewhat reduced for Bz-HC when compared to Hydrocodone.BT (the AUC value of hydrocodone for Bz-HC was approximately 72% of the AUC value for Hydrocodone.BT). The Cₘₐₓ value of hydrocodone for Bz-HC was approximately 92% of the Cₘₐₓ value for Hydrocodone.BT.

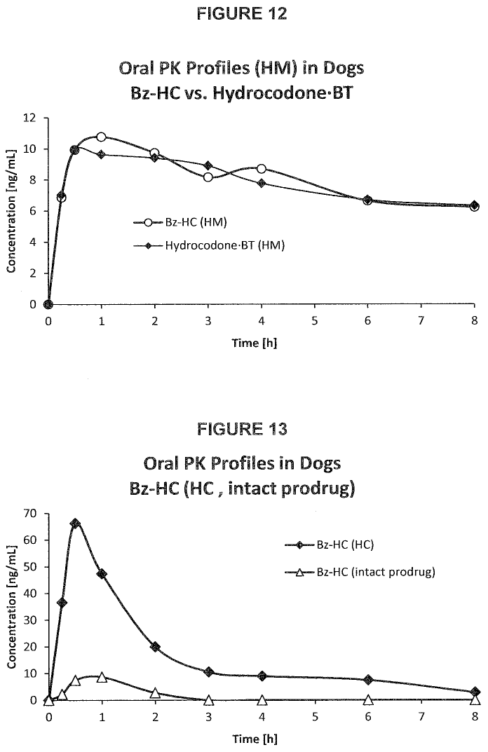

A comparison of the plasma concentrations of the active metabolite, hydromorphone, following oral administration of Bz-HC or Hydrocodone.BT is shown in FIG. 12. Systemic exposure and maximum plasma concentrations of hydromorphone were similar for both compounds. The AUC and Cₘₐₓ values of hydromorphone for Bz-HC were approximately 103% and 109% of the respective values for Hydrocodone.BT

A comparison the plasma concentrations of intact Bz-HC and hydrocodone released from Bz-HC is shown in FIG. 13. Similar to the results seen in rats, the plasma concentrations of intact Bz-HC prodrug in dogs were low when compared to the plasma concentrations of hydrocodone (the AUC value for intact Bz-HC was approximately 10% of the AUC value for hydrocodone).

### Example 10: Intravenous PK Profiles of Conjugated Hydrocodone, Hydrocodone, and Hydromorphone in Rats

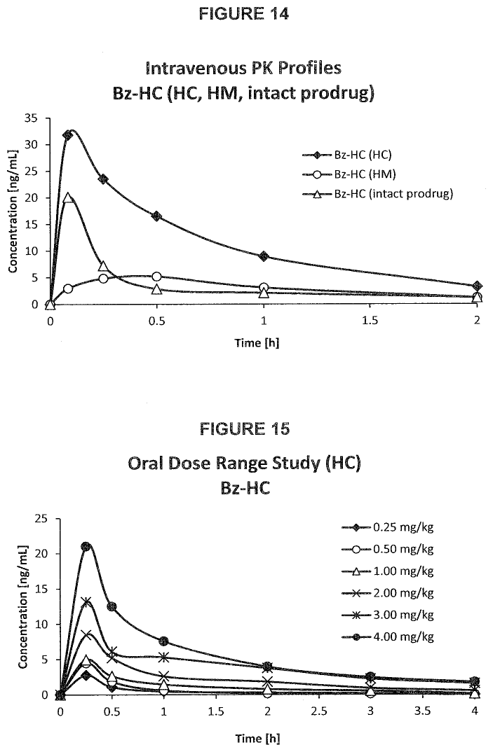

Bz-HC (0.30 mg/kg) was administered intravenously to rats. Due to its poor water solubility (or solubility in PBS), 0.30 mg/kg was close to the maximum dose that could be administered intravenously to rats. PK curves were determined for intact Bz-HC, hydrocodone, and the active metabolite hydromorphone. The plasma concentrations of intact Bz-HC, released hydrocodone, and the active metabolite, hydromorphone, were measured over time by LC-MS/MS. The resulting PK curves are shown in FIG. 14.

### Example 11: Oral PK Profiles of Hydrocodone and Hydromorphone Following Various Dosages of Bz-HC in Rats

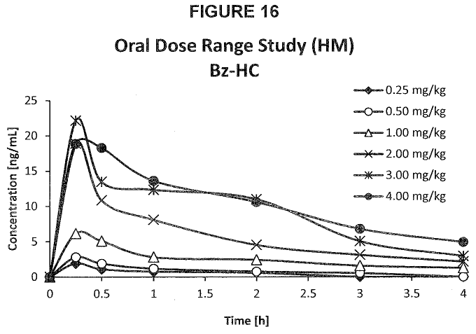

Bz-HC was orally administered to rats at dosages of 0.25, 0.50, 1.00, 2.00, 3.00, or 4.00 mg/kg. The plasma concentrations of hydrocodone or hydromorphone were measured by LC-MS/MS, as demonstrated in FIGS. 15 and 16, respectively. The exposures (AUC) to hydrocodone and hydromorphone at doses of Bz-HC between 0.25 and 4.00 mg/kg were fairly linear. The respective Cₘₐₓ values, however, were more variable, particularly for hydromorphone. The maximum plasma concentrations of hydromorphone did not significantly change at doses above 2.00 mg/kg of Bz-HC.

### Example 12: Intranasal Pharmacokinetic Study

Certain prodrug conjugates of the present technology were dosed as intranasal solutions in rats and compared to an equimolar solution of hydromorphone hydrochloride. The intranasal studies were performed at doses equimolar to 2.0 mg/kg of hydromorphone. The release of hydromorphone from the prodrugs varied depending on the ligand attached to hydromorphone.

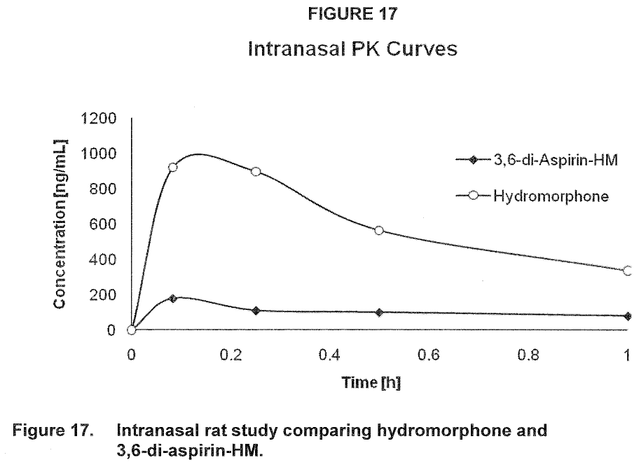

Plasma concentrations of hydromorphone after intranasal administration of 3,6-di-aspirin-HM were significantly reduced when compared to the parent drug (FIG. 17). The AUC and Cₘₐₓ values of 3,6-di-aspirin-HM were 17% and 20% of the respective PK parameters of unconjugated hydromorphone.

### Example 13: Intravenous Pharmacokinetic Study

Certain prodrug conjugates of the present technology were dosed as intravenous solutions in rats and compared to an equimolar solution of hydromorphone hydrochloride. The release of hydromorphone from the prodrugs varied depending on the ligand attached to hydromorphone.

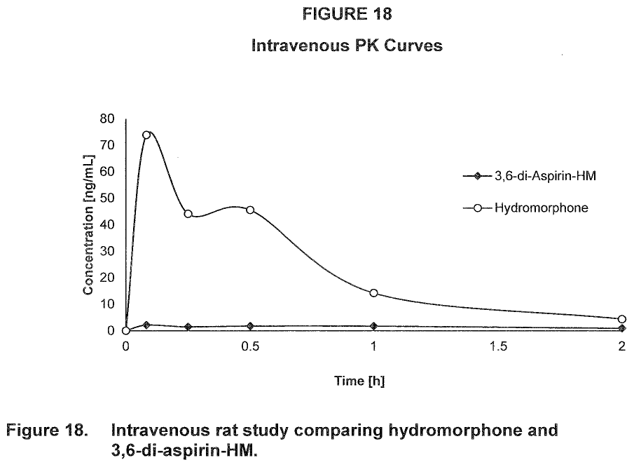

Hydromorphone and 3,6-di-aspirin-HM were dosed intravenously in rats at 0.20 mg/kg. Plasma concentrations of hydromorphone after intravenous administration of 3,6-di-aspirin-HM were significantly lower when compared to unconjugated hydromorphone (FIG. 18). The AUC and Cₘₐₓ values of 3,6-di-aspirin-HM were 6% and 3% of the respective PK parameters of unconjugated hydromorphone.

### Example 14: Dose Escalation Study

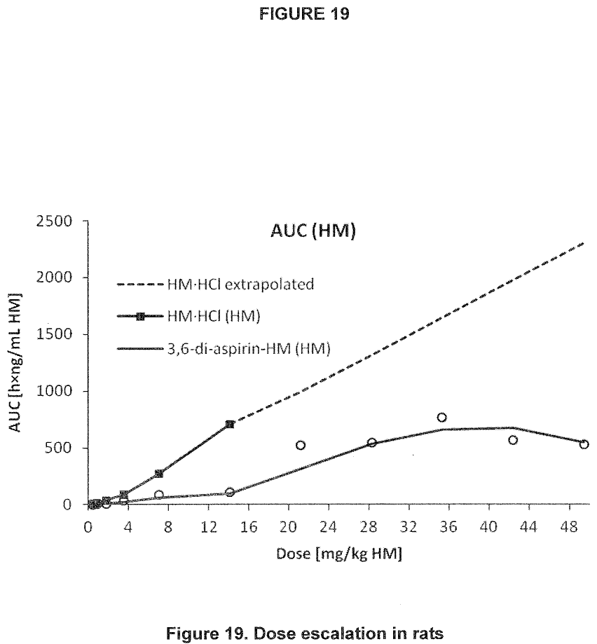

Certain prodrug conjugates of the present technology were dosed at escalating dosages as oral solutions in rats. When 3,6-di-aspirin-HM was dosed above the therapeutic level, the exposure (AUC) to hydromorphone reached a plateau. However, after oral administration of hydromorphone hydrochloride, the exposure (AUC) to hydromorphone remained approximately dose proportional even above the therapeutic level and caused death of the test animals with dosages above 14 mg/kg (see FIG. 19). These data suggest that 3,6-di-aspirin-HM has a decreased potential for causing overdose when compared to hydromorphone hydrochloride.

Without being bound by theory, it is believed that the exposure (AUC) plateau seen when 3,6-di-aspirin-HM was dosed above the therapeutic level is due to saturation of hydrolytic enzymes.

### Example 15: Tamper Resistance Study

Certain prodrug conjugates of the present technology were exposed to various commonly applied “extraction methods” to test for hydrolysis and/or decomposition of the prodrug. Solvent extraction of 3,6-di-aspirin-HM from formulation only yielded inactive prodrug with inherent pharmacological abuse protection. This shows that hydromorphone cannot be released from 3,6-di-aspirin-HM through physical manipulation or solvent extraction. In addition, 3,6-di-aspirin-HM is chemically stable under commonly applied “extraction methods” and only hydrolyzed and/or decomposed under extremely harsh conditions yielding a complex mixture of decomposition products in highly acidic or caustic solutions. Additionally, the decomposition products exhibited reduced oral, IN and IV bioavailability making extraction inefficient and impractical. The results of the extraction study are summarized in Table 5 below.

TABLE 5
Release of hydromorphone from 3,6-di-aspirin-HM

| Condition | Ambient Temperature | |
|---|---|---|
| (Common Methods) | 30 min. | 60 min. |
| 1 N HCl | 0 | 0 |
| Glacial acetic acid | 0 | 0 |
| 5% Acetic acid | 0 | 0 |
| Water | 0 | 0 |
| Sat. NaHCO₃ | 0 | 0 |
| 1 N NaOH | 1% | 1% |
| 4 N NaOH | 1% | 6% |

Numbers represent amount of hydromorphone released from 3,6-di-aspirin-HM (as %-AUC by HPLC)

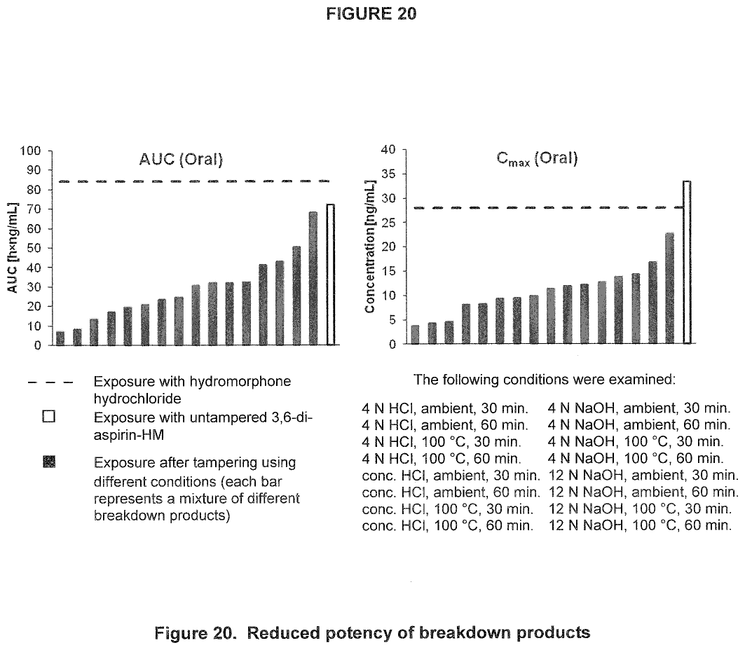

In addition, 3,6-di-aspirin-HM was exposed to 16 harsh, hydrolytic conditions and the resulting breakdown products were monitored and quantified by HPLC. Besides hydromorphone, three intermediate breakdown products were observed and then synthesized and dosed orally in rats. For each hydrolytic condition, virtual AUC and Cₘₐₓ values were calculated based on the composition of the observed mixture and on the individual PK parameters for each of its components (see FIG. 20). These data show that tampering with 3,6-di-aspirin-HM produces a mixture of compounds that when taken orally results in exposure (AUC) of hydromorphone that is lower than the exposure (AUC) seen with hydromorphone hydrochloride or untampered 3,6-di-aspirin-HM and in a maximum exposure (Cₘₐₓ) of hydromorphone that is lower than the maximum exposure (Cₘₐₓ) seen with hydromorphone hydrochloride.

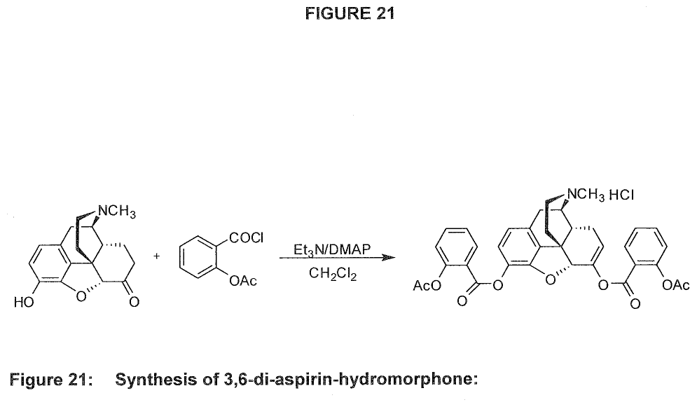

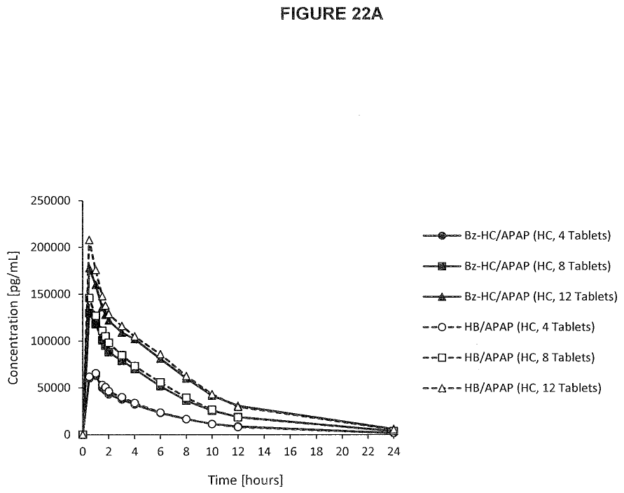

### Example 16: Synthesis of 3,6-Di-Aspirin-HM.HCl (FIG. 21)

Triethylamine (0.70 mL, 5 mmol) was added to hydromorphone hydrochloride (0.322 g, 1 mmol) in dichloromethane (15 mL) followed by DMAP (48.9 mg, 0.4 mmol) and O-acetylsalicyloyl chloride (0.794 g, 4 mmol). The reaction was stirred at room temperature for 48 hours. The mixture was poured into ethyl acetate (100 mL) and washed with aqueous saturated NaHCO₃ (30 mL×3) and brine (30 mL). The organic layer was dried over anhydrous Na₂SO₄ and concentrated. The residue was purified by column chromatography (ethyl acetate and then 8% methanol in dichloromethane) and subsequently further purified by PTLC (8% methanol in dichloromethane). The desired fraction was concentrated and converted to its HCl salt by adding 1 N HCl (1 mL). The solvent was evaporated and to the residue was added ether (15 mL). The resulting solid was collected and rinsed with ether (2 mL×3). The yield was 0.203 g (31.4%).

Description of Bioanalytical Methods Used in Example 17

Validated LC/MS/MS methods were used to measure plasma concentrations of Bz-HC, hydrocodone, hydromorphone and acetaminophen (APAP). The lower limits of quantitation (LLOQ) for Bz-HC, hydrocodone, hydromorphone, and APAP in plasma were 25 pg/mL, 250 pg/mL, 25 pg/mL, and 0.025 μg/mL, respectively.

Description of Pharmacokinetic and Statistical Analysis Conducted in Example 17

Actual blood sampling collection times were used in all PK analyses. Per protocol times were used to calculate mean plasma concentrations for graphical displays. Pharmacokinetic parameters for hydrocodone, hydromorphone, and APAP were calculated using standard equations for non-compartmental analysis. Only plasma concentrations that were greater than the LLOQs for the respective assays were used in the pharmacokinetic analysis.

### Example 17: Bz-HC.HCl/APAP Human Pharmacokinetic Studies

A study was conducted to assess the pharmacokinetics of Bz-HC, hydrocodone and hydromorphone after administration of single oral doses of Bz-HC.HCl/acetaminophen (APAP) tablets (6.67 mg/325 mg) and hydrocodone bitartrate (HB)/APAP (7.5 mg/325 mg) at three different dose levels (4, 8, and 8 tablets) under fasted conditions.

This was a single-center, randomized, double-blind, active- and placebo-controlled, and 7-period crossover. After completing an overnight fast (minimum 8 hours), subjects received each of the following 7 treatments according to their randomized treatment sequence:

A. 12 placebo capsules

B. 12 Bz HC.HCl/APAP 6.67 mg/325 mg tablets (over encapsulated) (80.04 mg Bz HC.HCl/3,900 mg acetaminophen)

C. 4 placebo capsules+8 Bz HC.HCl/APAP 6.67 mg/325 mg tablets (over encapsulated) (53.36 mg HC.HCl/APAP/2,600 mg acetaminophen)

D. 8 placebo capsules+4 Bz HC.HCl/APAP 6.67 mg/325 mg tablets (over encapsulated) (26.68 mg HC.HCl/APAP/1,300 mg acetaminophen)

E. 12 HB/APAP 7.5 mg/325 mg tablets (over encapsulated) (90 mg HB/3,900 mg acetaminophen)

F. 4 placebo capsules+8 HB/APAP 7.5 mg/325 mg tablets (over encapsulated) (60 mg HB/2,600 mg acetaminophen)

G. 8 placebo capsules+4 HB/APAP 7.5 mg/325 mg tablets (over encapsulated) (30 mg HB/1,300 mg acetaminophen)

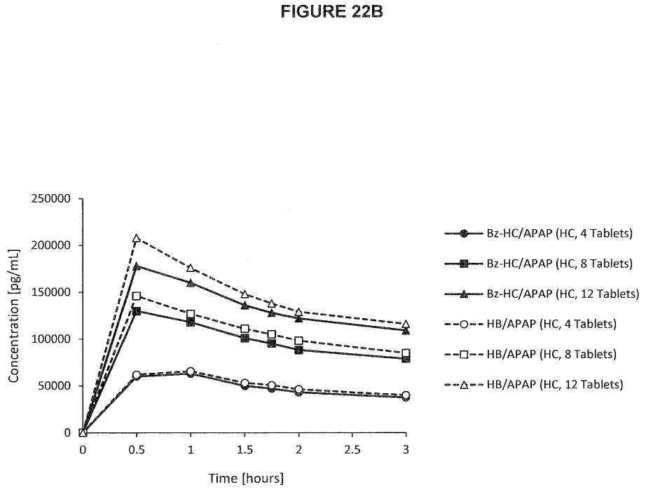

On dosing days blood samples were collected for Bz-HC.HCl, hydrocodone, and hydromorphone analysis at the following sampling times: within 1 hour predose and at 0.5, 1, 1.5, 1.75, 2, 3, 4, 6, 8, 10, 12, and 24 hour postdose. As shown in FIGS. 22a and 22b, at the low-dose (4 tablets, for example) and mid-dose (8 tablets, for example), the composition of Bz-HC.HCl/APAP 6.67 mg/325 mg provided a therapeutically bioequivalent AUC or Cₘₐₓ or both for hydrocodone at about lower than 53 mg when compared to an equivalent molar amount of unconjugated hydrocodone. At the high-dose (12 tablets, for example), the composition of Bz-HC.HCl/APAP 6.67 mg/325 mg exhibited an improved AUC and rate of release of hydrocodone over time when compared to unconjugated hydrocodone over the same time period. The composition at the high-dose exhibited lower exposure to hydrocodone at about more than 53 mg when compared to an equivalent molar amount of unconjugated hydrocodone. The composition at the high-dose also exhibited a lower peak exposure (Cₘₐₓ) to hydrocodone at about more than 53 mg when compared to an equivalent molar amount of unconjugated hydrocodone.

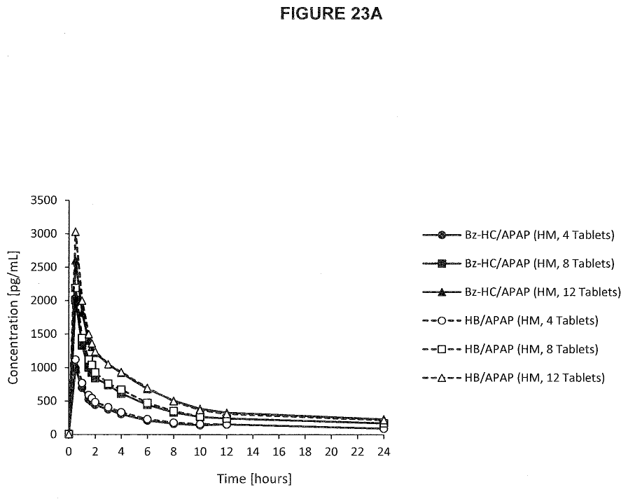

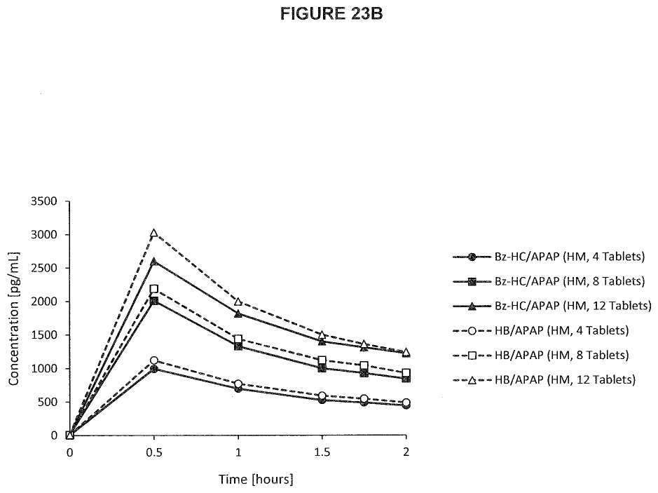

As shown in FIGS. 23a and 23b, at the low-dose (4 tablets, for example) and mid-dose (8 tablets, for example), the composition of Bz-HC.HCl/APAP 6.67 mg/325 mg provided a therapeutically bioequivalent AUC or Cₘₐₓ or both for hydromorphone at about lower than 53 mg when compared to an equivalent molar amount of unconjugated hydromorphone. At the high-dose (12 tablets, for example), the composition of Bz-HC.HCl/APAP 6.67 mg/325 mg exhibited an improved AUC and rate of release of hydromorphone over time when compared to unconjugated hydrocodone over the same time period. The composition at the high-dose also exhibited a lower exposure to hydromorphone at about more than 53 mg when compared to an equivalent molar amount of unconjugated hydrocodone. The composition at the high-dose also exhibited lower peak exposure (Cₘₐₓ) to hydromorphone at about more than 53 mg when compared to an equivalent molar amount of unconjugated hydrocodone.

A summary of the comparative PK data for Bz-HC.HCl/APAP and HB/APAP is presented in the following Table 6.

TABLE 6

| | Hydrocodone^{a} | | | Hydromorphone^{a} | | |
|---|---|---|---|---|---|---|
| PK | | 8 | 12 | | 8 | 12 |
| Parameter | 4Tablets | Tablets | Tablets | 4 Tablets | Tablets | Tablets |
| Cₘₐₓ | 96.4% | 90.2% | 90.8% | 88.8% | 91.2% | 87.5% |
| AUCₗₐₛₜ | 98.4% | 94.2% | 95.8% | 92.8% | 94.9% | 98.8% |
| AUC_{INF} | 98.2% | 94.8% | 97.1% | 107.0% | 99.2% | 107.6% |
| AUC_{0-0.5} | 96.9% | 88.7% | 86.1% | 88.5% | 92.0% | 86.4% |
| AUC₀₋₁ | 96.7% | 89.6% | 86.9% | 88.7% | 92.0% | 86.9% |
| AUC₀₋₂ | 95.5% | 90.7% | 89.4% | 89.1% | 91.4% | 89.7% |
| AUC₀₋₄ | 94.7% | 91.5% | 91.9% | 89.9% | 92.4% | 93.3% |
| AUC₀₋₈ | 95.6% | 92.2% | 93.3% | 90.0% | 92.4% | 95.0% |
| AUC₀₋₂₄ | 98.4% | 94.2% | 95.8% | 92.8% | 94.9% | 98.8% |

^{a}Entries represent the percent-ratio of the respective mean PK parameter for Bz-HC•HCl/APAP to the same mean PK parameter for HB/APAP. Percentages <100% indicate a lower value of the respective PK parameter for Bz-HC•HCl/APAP compared to HB/APAP.

Mean peak exposure to hydrocodone was lower with Bz HC.HCl/APAP at the mid- and high-dose but similar at the low-dose when compared to HB/APAP (Table 6). The ratio of mean Cₘₐₓ values for Bz-HC.HCl/APAP:HB/APAP in terms of hydrocodone exposure was greater than 96% at the low-dose, such as 4 tablets. The ratio of mean Cₘₐₓ values for Bz-HC.HCl/APAP:HB/APAP in terms of hydrocodone exposure was about 90-91% at the mid- and high-dose, such as 8 and 12 tablets.

Drug users seek fast onset of euphoria for fast reward which plays an important role in reinforcing behavior and addiction. As a result, lower opioid exposure, particularly in the first 1-2 hours following administration, is less desirable by drug users and more desirable for abuse-deterrent opioid therapies. At the mid- and high-dose, the partial areas under the curve for hydrocodone from 0 to 0.5 hours post-dose (AUC_{0-0.5}), from 0 to 1 hour post-dose (AUC₀₋₁), and from 0 to 2 hours post-dose (AUC₀₋₂) showed the most significant reduction in exposure with Bz-HC.HCl APAP compared to HB/APAP (Table 6).

### Example 18: Preparation of Composition Comprising Benzhydrocodone Conjugate or Asalhydromorphone

A direct compression benzhydrocodone formulation, for an immediate release opioid analgesic was formed by weighing each component separately and pre-screening with serial dilution portions of the formulation to improve API homogeneity due to low drug load in the blend. API with filler was mixed first in a V-blender for 100 revolutions, before the gelling agent was added with additional filler and mixed additionally for 100 revolutions. The other remaining excipients were added to the above blend except for the lubricant and mixed additionally at the same rate for 250 revolutions. Finally, the lubricant was added to the formulation and blended at the same rate for an additional 75 revolutions. This blend was further compressed on a rotary tablet press to form pharmaceutically acceptable tablets.

Table 7 shows representative immediate release formulations comprising 10 mg of benzhydrocodone conjugate, made according to the Example 18 procedure:

TABLE 7
10 mg benzhydrocodone formulations

| | Formulation | | | | | | | | | |
|---|---|---|---|---|---|---|---|---|---|---|
| | 1 | | 2 | | 3 | | 4 | | 5 | |
| | % | mg/ | % | mg/ | % | mg/ | % | mg/ | % | mg/ |
| | (w/w) | Tablet | (w/w) | Tablet | (w/w) | Tablet | (w/w) | Tablet | (w/w) | Tablet |
| API | 2.59 | 10 | 2.13 | 10 | 1.67 | 10 | 2.52 | 10 | 2.07 | 10 |
| PEO | 6.48 | 25 | 5.32 | 25 | 4.17 | 25 | 7.56 | 30 | 6.20 | 30 |
| MCC | 59.59 | 230 | 66.81 | 314 | 74.00 | 444 | 59.45 | 236 | 66.74 | 323 |
| Crospovidone | 25.39 | 98 | 20.85 | 98 | 16.33 | 98 | 24.69 | 98 | 20.25 | 98 |
| SLS | 5.18 | 20 | 4.26 | 20 | 3.33 | 20 | 5.04 | 20 | 4.13 | 20 |
| Mg Stearate | 0.26 | 1 | 0.21 | 1 | 0.17 | 1 | 0.25 | 1 | 0.21 | 1 |
| Cab-O-Sil | 0.52 | 2 | 0.43 | 2 | 0.33 | 2 | 0.50 | 2 | 0.41 | 2 |
| Total | 100.00 | 386 | 100.00 | 470 | 100.00 | 600 | 100.00 | 397 | 100.00 | 484 |

| | Formulation | | | | | | | |
|---|---|---|---|---|---|---|---|---|
| | 6 | | 7 | | 8 | | 9 | |
| | % (w/w) | mg/Tablet | % (w/w) | mg/Tablet | % (w/w) | mg/Tablet | % (w/w) | mg/Tablet |
| API | 1.62 | 10 | 2.44 | 10 | 2.01 | 10 | 1.57 | 10 |
| PEO | 4.86 | 30 | 8.56 | 35 | 7.03 | 35 | 5.51 | 35 |
| MCC | 73.91 | 456 | 59.41 | 243 | 66.67 | 332 | 73.86 | 469 |
| Crospovidone | 15.88 | 98 | 23.96 | 98 | 19.68 | 98 | 15.43 | 98 |
| SLS | 3.24 | 20 | 4.89 | 20 | 4.02 | 20 | 3.15 | 20 |
| Mg Stearate | 0.16 | 1 | 0.24 | 1 | 0.20 | 1 | 0.16 | 1 |
| Cab-O-Sil | 0.32 | 2 | 0.49 | 2 | 0.40 | 2 | 0.31 | 2 |
| Total | 100.00 | 617 | 100.00 | 409 | 100.00 | 498 | 100.00 | 635 |
| | Formulation | | | | | | | |
| | 11 | | 12 | | 13 | | 14 | |
| | % (w/w) | mg/Tablet | % (w/w) | mg/Tablet | % (w/w) | mg/Tablet | % (w/w) | mg/Tablet |
| API | 4.77 | 10 | 5.57 | 10 | 3.69 | 10 | 3.73 | 10 |
| PEO | 5.97 | 12.5 | 6.96 | 12.5 | 9.23 | 25 | 9.33 | 25 |
| MCC | 54.89 | 115 | 47.35 | 85 | 42.44 | 115 | 55.97 | 150 |
| Crospovidone | 23.39 | 49 | 27.30 | 49 | 36.16 | 98 | 22.39 | 60 |
| SLS | 9.55 | 20 | 11.14 | 20 | 7.38 | 20 | 7.46 | 20 |
| Mg Stearate | 0.48 | 1 | 0.56 | 1 | 0.37 | 1 | 0.37 | 1 |
| Cab-O-Sil | 0.95 | 2 | 1.11 | 2 | 0.74 | 2 | 0.75 | 2 |
| Total | 100.00 | 209.5 | 100.00 | 179.5 | 100.00 | 271 | 100.00 | 268 |

Table 8 shows representative immediate release formulations comprising 30 mg of benzhydrocodone conjugate, made according to the Example 18 procedure.

TABLE 8
30 mg benzhydrocodone formulations

| | Formulation | | | | | | | | | |
|---|---|---|---|---|---|---|---|---|---|---|
| | DOE 1 | | DOE 2 | | DOE 3 | | DOE 4 | | DOE 5 | |
| | % | mg/ | % | mg/ | % | mg/ | % | mg/ | % | mg/ |
| | (w/w) | Tablet | (w/w) | Tablet | (w/w) | Tablet | (w/w) | Tablet | (w/w) | Tablet |
| API | 7.39 | 30 | 6.12 | 30 | 4.84 | 30 | 7.19 | 30 | 5.95 | 30 |
| PEO | 6.16 | 25 | 5.10 | 25 | 4.03 | 25 | 7.19 | 30 | 5.95 | 30 |
| MCC | 56.65 | 230 | 64.08 | 314 | 71.61 | 444 | 56.59 | 236 | 64.09 | 323 |
| Crospovidone | 24.14 | 98 | 20.00 | 98 | 15.81 | 98 | 23.50 | 98 | 19.44 | 98 |
| SLS | 4.93 | 20 | 4.08 | 20 | 3.23 | 20 | 4.80 | 20 | 3.97 | 20 |
| Mg Stearate | 0.25 | 1 | 0.20 | 1 | 0.16 | 1 | 0.24 | 1 | 0.20 | 1 |
| Cab-O-Sil | 0.49 | 2 | 0.41 | 2 | 0.32 | 2 | 0.48 | 2 | 0.40 | 2 |
| Total | 100.00 | 406 | 100.00 | 490 | 100.00 | 620 | 100.00 | 417 | 100.00 | 504 |

| | Formulation | | | | | | | |
|---|---|---|---|---|---|---|---|---|
| | DOE 6 | | DOE 7 | | DOE 8 | | DOE 9 | |
| | % (w/w) | mg/Tablet | % (w/w) | mg/Tablet | % (w/w) | mg/Tablet | % (w/w) | mg/Tablet |
| API | 4.71 | 30 | 6.99 | 30 | 5.79 | 30 | 4.58 | 30 |
| PEO | 4.71 | 30 | 8.16 | 35 | 6.76 | 35 | 5.34 | 35 |
| MCC | 71.59 | 456 | 56.64 | 243 | 64.09 | 332 | 71.60 | 469 |
| Crospovidone | 15.38 | 98 | 22.84 | 98 | 18.92 | 98 | 14.96 | 98 |
| SLS | 3.14 | 20 | 4.66 | 20 | 3.86 | 20 | 3.05 | 20 |
| Mg Stearate | 0.16 | 1 | 0.23 | 1 | 0.19 | 1 | 0.15 | 1 |
| Cab-O-Sil | 0.31 | 2 | 0.47 | 2 | 0.39 | 2 | 0.31 | 2 |
| Total | 100.00 | 637 | 100.00 | 429 | 100.00 | 518 | 100.00 | 655 |
| | Formulation | | | | | | | |
| | DOE 11 | | DOE 12 | | DOE 13 | | DOE 14 | |
| | % (w/w) | mg/Tablet | % (w/w) | mg/Tablet | % (w/w) | mg/Tablet | % (w/w) | mg/Tablet |
| API | 13.16 | 30 | 15.15 | 30 | 10.36 | 30 | 10.47 | 30 |
| PEO | 5.48 | 12.5 | 6.31 | 12.5 | 8.64 | 25 | 8.73 | 25 |
| MCC | 50.44 | 115 | 42.93 | 85 | 39.72 | 115 | 52.36 | 150 |
| Crospovidone | 21.49 | 49 | 24.75 | 49 | 33.85 | 98 | 20.94 | 60 |
| SLS | 8.77 | 20 | 10.10 | 20 | 6.91 | 20 | 6.98 | 20 |
| Mg Stearate | 0.22 | 0.5 | 0.25 | 0.5 | 0.17 | 0.5 | 0.17 | 0.5 |
| Cab-O-Sil | 0.44 | 1 | 0.51 | 1 | 0.35 | 1 | 0.35 | 1 |
| Total | 100.00 | 228 | 100.00 | 198 | 100.00 | 290 | 100.00 | 287 |

Representative prophetic benzhydrocodone formulations are shown in Table 9 and are made according to the Example 18 procedure.

TABLE 9
Benzhydrocodone formulations (Prophetic):

| | Formulation - conjugate to PEO | | | | | | | | | |
|---|---|---|---|---|---|---|---|---|---|---|
| | 1:5 (1:5) | | 1:6 (1:6) | | 1:4.5 (1:4) | | 1:4 (1:4) | | 1:1 (1:1) | |
| | % | mg/ | % | mg/ | % | mg/ | % | mg/ | % | mg/ |
| API/PEO wt/wt | (w/w) | Tablet | (w/w) | Tablet | (w/w) | Tablet | (w/w) | Tablet | (w/w) | Tablet |
| API (mg | 1.54 | 11 (10) | 1.43 | 11 (10) | 2.00 | 11 (10) | 1.54 | 11 (10) | 5.00 | 22 (20) |
| conjugate base) | | | | | | | | | | |
| PEO | 7.69 | 50 | 8.57 | 60 | 9.00 | 45 | 6.15 | 40 | 5.00 | 20 |
| MCC | 68.00 | 442 | 68.86 | 482 | 64.80 | 324 | 73.69 | 479 | 59.75 | 239 |
| Crospovidone | 19.23 | 125 | 17.86 | 125 | 19.60 | 98 | 15.08 | 98 | 24.50 | 98 |
| SLS | 3.08 | 20 | 2.86 | 20 | 4.00 | 20 | 3.08 | 20 | 5.00 | 20 |
| Mg Stearate | 0.15 | 1 | 0.14 | 1 | 0.20 | 1 | 0.15 | 1 | 0.25 | 1 |
| Cab-O-Sil | 0.31 | 2 | 0.29 | 2 | 0.40 | 2 | 0.31 | 2 | 0.50 | 2 |
| Total | 100.00 | 650 | 100.00 | 700 | 100.00 | 500 | 100.00 | 650 | 100.00 | 400 |

| Formulation - conjugate to PEO | |
|---|---|
| 3:2 (3:2) | 2:1 (2:1) |

| | % (w/w) | mg/Tablet | % (w/w) | mg/Tablet |
|---|---|---|---|---|
| API (mg | 7.32 | 33 (30) | 14.96 | 33 (30) |
| conjugate base) | | | | |
| PEO | 4.88 | 20 | 7.48 | 15 |
| MCC | 58.29 | 229 | 42.39 | 85 |
| Crospovidone | 23.90 | 98 | 24.44 | 49 |
| SLS | 4.88 | 20 | 9.98 | 20 |
| Mg Stearate | 0.24 | 1 | 0.25 | 0.5 |
| Cab-O-0.49 | 2 | 0.50 | 1 | |
| Total | 100.00 | 400 | 100.00 | 200.5 |

### Example 19: Dissolution Study

Benzhydrocodone (30 mg) Formulations DOE 1-9 shown in Table 8 were evaluated for dissolution properties under the following dissolution conditions (discriminating dissolution method):

| | Dissolution Conditions | | |
|---|---|---|---|
| Apparatus: | 2 (rotating paddles) | Padde RPM: | 50, 200 (infinity) |
| Media | 0.1N HCl 0.01% CTAB | Temp: | 37 C. |
| Volume: | 900 mL | Pull Time: | 5, 10, 15, 20, 30, |
| | | | 60 min |
| sample vol: | 10 mL | sinker: | 2S |

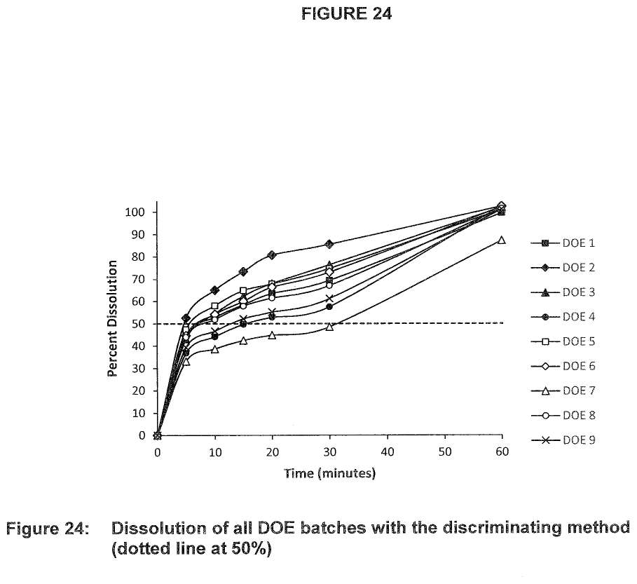

The results are shown graphically in FIG. 24 and demonstrate the effects of the various tablet formulations on the observed dissolution rates when utilizing the discriminatory dissolution conditions.

Benzhydrocodone (30 mg) Formulation 1 shown in Table 8 was evaluated for dissolution using a release dissolution method. The conditions for this method are shown in the following Table:

| | Dissolution Conditions | | |
|---|---|---|---|
| Apparatus: | 2 (rotating paddles) | Padde RPM: | 50, 200 (infinity) |
| Media | 0.1N HCl 0.01% CTAB | Temp: | 37 C. |
| Volume: | 900 mL | Pull Time: | 5, 10, 15, 20, 30, |
| | | | 60 min |
| sample vol: | 10 mL | sinker: | 4S |

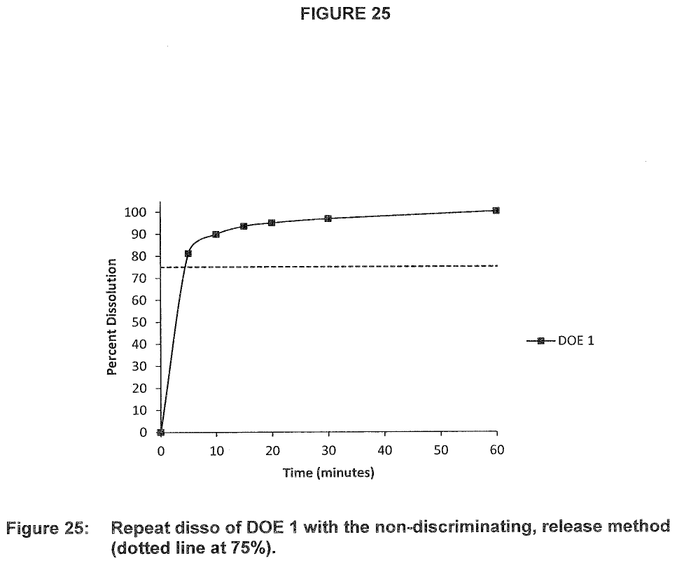

The results are shown graphically in FIG. 25 and illustrate an immediate release dissolution profile obtained using the non-discriminatory dissolution conditions. Using these conditions, the formulation used in DOE1 tablets release about 80% of KP201 within 10 minutes.

### Example 20: Preparation of Composition Comprising Benzhydrocodone Conjugate and Acetaminophen

Two wet-granulation processes have been used for formulations comprising the combination of benzhydrocodone and acetaminophen.

Method 1:

A wet granulated formulation shown in Table 10 for an immediate release opioid analgesic was formed by weighing each component and loading conjugate, microcrystalline cellulose, polymer (binder), crospovidone, and half the SLS to the high shear granulator and granulated by spraying water. The wet mass was subsequently transferred to a fluid bed dryer and milled to deagglomerate the mass. The wet mass was dried and then dry milled. The COMPAP L (granulated acetaminophen) and remaining SLS were loaded into a V-shell blender and blended for 5 minutes. Half of this blend was discharged, then the granulation was added to the V-shell blender and the discharged material was placed back on top. All materials were blended for 10 minutes. Screened lubricant was added to the V-blender and further blended for 3 additional minutes. This final blend was further compressed on a rotary tablet press and pharmaceutically acceptable tablets were formed.

TABLE 10

| Component | Weight (mg)/tablet |
|---|---|
| Bz-HC | 8.9 |
| COMPAP L | 361.1 |
| AvicelPH 102 | 331.0 |
| Polyox | 26 |
| Sodium lauryl sulfate | 20 |
| Crospovidone | 100 |
| Magnesium stearate | 3 |
| Total | 850 |

Method 2:

A wet granulated formulation as shown in Table 11 for an immediate release opioid analgesic was formed by two separate granulations. The first granulation loaded conjugate, 75% of the microcrystalline cellulose, 50% of the polymer (binder), 50% of the SLS and 45% crospovidone to the high shear granulator and granulated by spraying water. The wet mass was subsequently transferred to a fluid bed dryer and milled to deagglomerate the mass. The wet mass was dried and then dry milled. The second granulation loaded APAP powder, and remaining 50% of polymer (binder) and 45% crospovidone to the high shear granulator and granulated by spraying water. The wet mass was subsequently transferred to a fluid bed dryer and milled to deagglomerate the mass. The wet mass was dried and then dry milled. The remaining microcrystalline cellulose, crospovidone and SLS were loaded to a V-blender and blended for 5 minutes. Discharged half of this blend and added the granulations to the V-blender placing the discharged material back on top. All the materials were blended for 15 minutes. Screened lubricant was added to the V-blender and the materials were blended for 3 additional minutes. This final blend was further compressed on a rotary tablet press where tablets were unable to reach targeted hardness and were unacceptable.

TABLE 11

| Component | Weight (mg)/tablet |
|---|---|
| Bz-HC | 8.9 |
| APAP | 325 |
| Polyox | 26 |
| Avicel PH 102 | 166.2 |
| Sodium lauryl sulfate | 20 |
| Crospovidone | 100 |
| Stearic Acid | 3.9 |
| Total | 650 |

Two Granulations (Prophetic):

Wet granulation of benzhydrocodone using polyethylene oxide as the binder, wet granulation of APAP using polyethylene oxide as the binder.

A wet granulated formulation as shown in Table 12 (prophetic) for an immediate release opioid analgesic is formed by two separate granulations. The first granulation loads conjugate, 75% of the microcrystalline cellulose, 50% of the polymer (binder), 50% of the SLS and 45% crospovidone to the high shear granulator and the materials are granulated by spraying water. The wet mass is subsequently transferred to a fluid bed dryer and milled to deagglomerate the mass. The wet mass is dried and then dry milled. The second granulation loads APAP powder, and remaining 50% of polymer (binder) and 45% crospovidone, remaining microcrystalline cellulose and povidone to the high shear granulator and the materials are granulated by spraying water. The wet mass is subsequently transferred to a fluid bed dryer and milled to deagglomerate the mass. The wet mass is dried and then dry milled. The remaining crospovidone and SLS are loaded to a V-blender and blended for 5 minutes. Half of this blend is discharged and then the granulations are added to the V-blender placing the discharged material back on top. All the materials are blended for 15 minutes. Screened lubricant is added to the V-blender and blending is continued for 3 additional minutes. This final blend is further compressed on a rotary tablet press to form expected pharmaceutically acceptable tablets.

TABLE 12

| Component | Weight (mg)/tablet |
|---|---|
| Bz-HC | 8.9 |
| APAP | 325 |
| AvicelPH 102 | 217.1 |
| Polyox | 26 |
| Sodium lauryl sulfate | 20 |
| Crospovidone | 100 |
| Magnesium stearate | 3 |
| Total | 700 |

Additional prophetic immediate release formulations comprising benzhydrocodone conjugate and acetaminophen are shown in Table 13.

TABLE 13
Benzhydrocodone/APAP formulations (Prophetic):

| Formulation - Conjugate+APAP to PEO Ratio | | | |
|---|---|---|---|
| 24:1 (24:1) | 20:1 (20:1) | 12:1 (12:1) | 15:1 (15:1) |

| | % | mg/ | % | mg/ | % | mg/ | % | mg/ |
|---|---|---|---|---|---|---|---|---|
| | (w/w) | Tablet | (w/w) | Tablet | (w/w) | Tablet | (w/w) | Tablet |
| API (mg | 0.66 | 4.45 | 0.74 | 4.45 (4.1) | 1.27 | 8.9 (8.1) | 0.74 | 4.45 (4.1) |
| conjugate | | | | | | | | |
| base) | | | | | | | | |
| APAP | 48.15 | 325 | 54.17 | 325 | 46.43 | 325 | 54.17 | 325 |
| Povidone | 1.48 | 10 | 1.67 | 10 | 0.43 | 3 | 1.67 | 10 |
| PEO | 2.07 | 14 | 2.75 | 16.5 | 4.00 | 28 | 3.67 | 22 |
| MCC | 24.08 | 162.5 | 20.84 | 125.05 | 30.16 | 211.1 | 19.26 | 115.55 |
| Crospovidone | 18.52 | 125 | 15.83 | 95 | 14.29 | 100 | 16.50 | 99 |
| SLS | 4.44 | 30 | 3.33 | 20 | 2.86 | 20 | 3.33 | 20 |
| Stearic Acid | 0.59 | 4 | 0.67 | 4 | 0.57 | 4 | 0.67 | 4 |
| Total | 100.00 | 675 | 100.00 | 600 | 100.00 | 650 | 100.00 | 600 |

| Formulation - Conjugate+APAP to PEO Ratio | | |
|---|---|---|
| 10:1 (10:1) | 7.5:1 (7.5:1) | 6:1 (6:1) |

| | % | mg/ | % | mg/ | % | mg/ |
|---|---|---|---|---|---|---|
| | (w/w) | Tablet | (w/w) | Tablet | (w/w) | Tablet |
| API (mg | 1.48 | 8.9 (8.1) | 1.27 | 8.9 (8.1) | 1.24 | 8.9 |
| conjugate | | | | | | |
| base) | | | | | | |
| APAP | 54.17 | 325 | 46.43 | 325 | 45.45 | 325 |
| Povidone | 1.50 | 9 | 1.43 | 10 | 1.40 | 10 |
| PEO | 5.67 | 34 | 6.43 | 45 | 7.69 | 55 |
| MCC | 16.68 | 100.1 | 23.16 | 162.1 | 22.67 | 162.1 |
| Crospovidone | 16.50 | 99 | 17.86 | 125 | 17.48 | 125 |
| SLS | 3.33 | 20 | 2.86 | 20 | 3.50 | 25 |
| Stearic Acid | 0.67 | 4 | 0.57 | 4 | 0.56 | 4 |
| Total | 100.00 | 600 | 100.00 | 700 | 100.00 | 715 |

| | Formulation - Conjugate Ratio | | | | | | | | | |
|---|---|---|---|---|---|---|---|---|---|---|
| | 1:10 (1:11) | | 1:8 (1:9) | | 2:5 (1:3) | | 1:2 (1:2) | | 1:3 (1:3) | |
| | % | mg/ | % | mg/ | % | mg/ | % | mg/ | % | mg/ |
| | (w/w) | Tablet | (w/w) | Tablet | (w/w) | Tablet | (w/w) | Tablet | (w/w) | Tablet |
| API | 0.64 | 4.45 (4.1) | 0.64 | 4.45 (4.1) | 1.27 | 8.9 (8.1) | 137 | 8.9 (8.1) | 1.03 | 6.67(6.1) |
| APAP | 46.43 | 325 | 46.43 | 325 | 46.43 | 325 | 50.00 | 325 | 50.00 | 325 |
| Povidone | 0.00 | 0 | 0.71 | 5 | 0.71 | 5 | 1.54 | 10 | 1.54 | 10 |
| PEO | 6.36 | 44.5 | 5.14 | 36 | 3.18 | 22.25 | 2.74 | 17.8 | 3.08 | 20 |
| MCC | 28.86 | 202.05 | 29.36 | 205.55 | 30.69 | 214.85 | 25.28 | 164.3 | 25.28 | 164.33 |
| Crospovidone | 14.29 | 100 | 14.29 | 100 | 14.29 | 100 | 15.38 | 100 | 15.38 | 100 |
| SLS | 2.86 | 20 | 2.86 | 20 | 2.86 | 20 | 3.08 | 20 | 3.08 | 20 |
| Stearic Acid | 0.57 | 4 | 0.57 | 4 | 0.57 | 4 | 0.62 | 4 | 0.62 | 4 |
| Total | 100.00 | 700 | 100.00 | 700 | 100.00 | 700 | 100.00 | 650 | 100.00 | 650 |

Preparation of Composition Comprising Asalhydromorphone Conjugate (Prophetic)

The Example 18 procedure is used to form a direct compression asalhydromorphone formulation, for an immediate release opioid analgesic.

Representative prophetic immediate release formulations comprising the asalhydromorphone conjugate are shown in Table 14.

TABLE 14
3,6-di-aspirin-hydromorphone formulations (Prophetic):

| | Formulation - conjugate to PEO | | | | | | | | | |
|---|---|---|---|---|---|---|---|---|---|---|
| | 1:10 (1:10) | | 1:9 (1:9) | | 1:8 (1:8) | | 1:5 (1:5) | | 1:2 (1:2) | |
| | % | mg/ | % | mg/ | % | mg/ | % | mg/ | % | mg/ |
| API/PEO wt/wt | (w/w) | Tablet | (w/w) | Tablet | (w/w) | Tablet | (w/w) | Tablet | (w/w) | Tablet |
| API (mg | 0.62 | 4 (3.8) | 0.62 | 4 (3.8) | 0.8 | 4 (3.8) | 1.23 | 8 (7.6) | 4.00 | 16 (15.1) |
| conjugate base) | | | | | | | | | | |
| PEO | 6.15 | 40 | 5.54 | 36 | 6.4 | 32 | 6.15 | 40 | 8.00 | 32 |
| MCC | 74.62 | 485 | 71.08 | 462 | 68.60 | 343 | 74.00 | 481 | 57.75 | 231 |
| Crospovidone | 15.08 | 98 | 19.23 | 125 | 19.60 | 98 | 15.08 | 98 | 24.50 | 98 |
| SLS | 3.08 | 20 | 3.08 | 20 | 4.00 | 20 | 3.08 | 20 | 5.00 | 20 |
| Mg Stearate | 0.15 | 1 | 0.15 | 1 | 0.20 | 1 | 0.15 | 1 | 0.25 | 1 |
| Cab-O-Sil | 0.31 | 2 | 0.31 | 2 | 0.40 | 2 | 0.31 | 2 | 0.50 | 2 |
| Total | 100.00 | 650 | 100.00 | 650 | 100.00 | 500 | 100.00 | 650 | 100.00 | 400 |

| Formulation - conjugate to PEO | | |
|---|---|---|
| 3:2 (3:2) | 3:1 (3:1) | 2:1 (2:1) |

| | % | mg/ | % | mg/ | % | mg/ |
|---|---|---|---|---|---|---|
| | (w/w) | Tablet | (w/w) | Tablet | (w/w) | Tablet |
| API (mg | 8.00 | 16 (15.1) | 8.00 | 16 (15.1) | 5.33 | 16 (15.1) |
| conjugate base) | | | | | | |
| PEO | 5.50 | 11 | 3.00 | 6 | 2.67 | 8 |
| MCC | 51.25 | 102.5 | 53.75 | 107.5 | 68.50 | 205.5 |
| Crospovidone | 24.50 | 49 | 24.50 | 49 | 16.33 | 49 |
| SLS | 10.00 | 20 | 10.00 | 20 | 6.67 | 20 |
| Mg Stearate | 0.25 | 0.5 | 0.25 | 0.5 | 0.17 | 0.5 |
| Cab-O-Sil | 0.50 | 1 | 0.50 | 1 | 0.33 | 1 |
| Total | 100.00 | 200 | 100.00 | 200 | 100.0 | 300 |

In the present specification, it should be appreciated by those of ordinary skill in the art that the use of the singular includes the plural except where specifically indicated. Additionally, the presently described technology is now described in such full, clear, concise and exact terms as to enable any person skilled in the art to which it pertains, to practice the same. It is to be understood that the foregoing describes preferred embodiments of the technology and that modifications may be made therein without departing from the spirit or scope of the invention as set forth in the appended claims.

1-203. (canceled)

204. A composition comprising:
at least one compound selected from the group consisting of asalhydromorphone having the following structure: 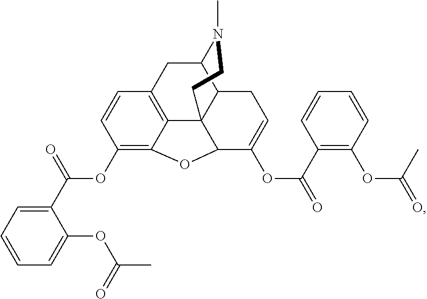
benzhydrocodone having the follow structure: 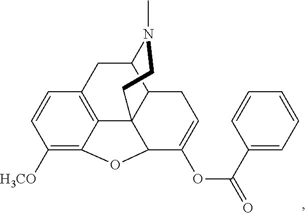
and pharmaceutically acceptable salts of said at least one compound;
at least one gel forming polymer selected from the group consisting of polyethylene oxide, hydroxypropyl methyl cellulose, carbomers, and combinations thereof;
at least one disintegrant selected from the group consisting of crospovidone, sodium starch glycolate, croscarmellose sodium, and combinations thereof;
and at least one surfactant selected from the group consisting of sodium lauryl sulfate, poloxamer, sorbitan monoesters, glyceryl monooleates, and combinations thereof.

205. The composition of claim 204, where in the pharmaceutically acceptable salt is selected from the group consisting of acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, l-lactate, d,l-lactate, d,l-malate, l-malate, d-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsufate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate (mucate), galacturonate, gallate, gentisate, glutamate, glutarate, glycerophosphate, heptanoate (enanthate), hydroxybenzoate, hippurate, phenylpropionate, iodide, xinafoate, lactobionate, laurate, maleate, mandelate, methanesufonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, undecylenate, sodium, potassium, calcium, magnesium, zinc, aluminum, lithium, cholinate, lysinium, ammonium, and tromethamine.

206. The composition of claim 205, wherein the pharmaceutically acceptable salt is a hydrochloride salt of asalhydromorphone having the following structure: 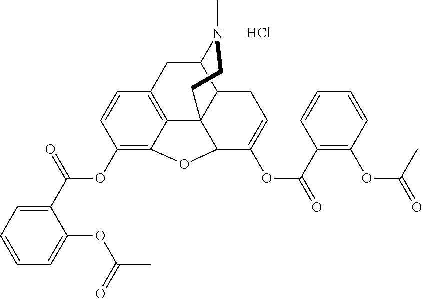
or a hydrochloride salt of benzhydrocodone having the following structure:

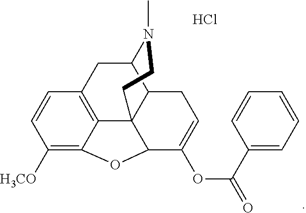

207. The composition of claim 204, wherein the at least one gel forming polymer is polyethylene oxide and further wherein the composition has a ratio of compound to polyethylene oxide of from about 1:10 to about 3:2 w/w %.

208. The composition of claim 204, wherein the at least one gel forming polymer is polyethylene oxide and further wherein the composition has a ratio of compound to polyethylene oxide of from about 1:5 to about 5:2 w/w %.

209. The composition of claim 204, wherein the at least one gel forming polymer is polyethylene oxide and further wherein the polyethylene oxide has an average molecular weight of about 900,000 to about 7,000,000.

210. The composition of claim 204, wherein the at least one disintegrant is crospovidone.

211. The composition of claim 210, wherein the crospovidone is provided in an amount of about 3 wt % to about 50 wt % of the composition.

212. The composition of claim 204, wherein the at least one surfactant is sodium lauryl sulfate.

213. The composition of claim 212, wherein the sodium lauryl sulfate is provided in an amount of about 1 wt % to about 20 wt % of the composition.

214. The composition of claim 204, wherein the composition is a pharmaceutical composition.

215. The composition of claim 214, wherein the pharmaceutical composition is provided in a unit dose form or a pharmaceutically acceptable dosage form.

216. The composition of claim 215, wherein the unit dose form or the pharmaceutically acceptable dosage form is selected from the group consisting of a powder, caplet, pill, suppository, gel, soft gelatin capsule, capsule, sachet, lozenge, troche, slurry, suspension, solution, oral film, and compressed tablet.

217. The composition of claim 214, wherein the composition is an immediate-release composition and/or has abuse deterrent properties.
